(19) United States
(12) Patent Application Publication
(10) Pub. No.: US 2017/0018201 A1
(43) Pub. Date: Jan. 19, 2017

(54) DATA PROCESSING DEVICE, DATA PROCESSING SYSTEM, TERMINAL DEVICE, DATA PROCESSING METHOD, AND DATA PROCESSING PROGRAM

(71) Applicant: SHARP KABUSHIKI KAISHA, Sakai City, Osaka (JP)

(72) Inventors: Daiki OGASAWARA, Sakai City, Osaka (JP); Hiroaki KONDO, Sakai City, Osaka (JP)

(73) Assignee: SHARP KABUSHIKI KAISHA, Sakai City, Osaka (JP)

(21) Appl. No.: 15/124,447

(22) PCT Filed: Apr. 1, 2015
(86) PCT No.: PCT/JP2015/060361

§ 371 Date: Sep. 8, 2016

(30) Foreign Application Priority Data
Apr. 2, 2014 (JP) 2014-076064

(51) Int. Cl. G09B 5/12; A61B 5/11; A61B 5/00; G06F 17/30; G09B 19/00

(52) U.S. Cl. CPC G09B 5/125; G06F 17/30864; G09B 19/003; G09B 19/0092; A61B 5/002; A61B 5/112; A61B 5/4806; A61B 5/486; A61B 5/4866; A61B 5/6802; A61B 5/6898

(57) ABSTRACT

A state information acquisition unit acquires state information indicating a state of a body, an information search unit searches for information on the state information from a content information storage unit that stores text information regarding broadcast content, and an output unit outputs information searched for by the information search unit.

### TECHNICAL FIELD

The present invention relates to a data processing device, a data processing system, a terminal device, a data processing method, and a data processing program, and particularly, to a data processing device, a data processing system, a terminal device, a data processing method, and a data processing program that are used in the field of healthcare.

Priority is claimed on Japanese Patent Application No. 2014-076064, filed Apr. 2, 2014, the content of which is incorporated herein by reference.

### BACKGROUND ART

Conventionally, a health management system that presents advice according to a state of a user using various measurement devices and a server device has been proposed. As a measurement device, a high-performance wearable motion measurement device such as a wristwatch type pedometer may be used. Measurement data in the measurement device includes the number of steps, a distance traveled, a sleep time, or the like. Further, a terminal device that enables data that cannot be measured by a measurement device, such as the content or the amount of ingested food to be input by an operation of a user may be used. A server device generates assessment results or advice on the basis of measurement data and input data, and presents the generated assessment results or advice to a user terminal. The assessment results include assessments that will help in health management, such as “excess calories”, “lack of exercise”, or “achievement of set target value”. The advice includes advice according to values of data, such as “Three hours of sleep is insufficient” or “Please additionally take in 40 g of protein”.

### CITATION LIST

### Patent Literature

PTL 1: Japanese Unexamined Patent Application, First Publication No. 2006-106952

### SUMMARY OF INVENTION

### Technical Problem

However, in a healthcare system of the related art, only quantitative advice is obtained. Therefore, the obtained advice tends to be monotonous. For example, means for achieving a predetermined target may not be obtained, or always only the same means may be obtained for the same measurement data or input data. Further, since the obtained advice is monotonous, the system may not be continuously used. In order to encourage use of the system, a function of providing a service of rewarding a user who has achieved a goal with a prize, points, or the like, or a service of encouraging competition between users such as sharing of data between specific users may be introduced. Even in such a case, since the advice is monotonous, the user may become bored with the service and the system may not be used or original advice may not be used. Accordingly, the service of a service company providing health advice may not be continuously used and, thus, the service company providing such a service may not obtain steady revenues.

The present invention has been made in view of the above points, and provides a data processing device in which provided information is continuously used, a data processing system, a terminal device, a data processing method, and a data processing program.

### Solution to Problem

(1) The present invention has been made to solve the above problems, and one aspect of the present invention is a data processing device, including: a state information acquisition unit that acquires state information indicating a state of a body; an information search unit that searches for information on broadcast content corresponding to the state information acquired by the state information acquisition unit in a content information storage unit that stores the information on the broadcast content; and an output unit that outputs information on the broadcast content searched for by the information search unit.

(2) In another aspect of the present invention, the data processing device of (1) further includes a keyword storage unit that stores keyword data including a keyword regarding state information indicating the state of the body; and a keyword determination unit that determines a keyword corresponding to the state information acquired by the state information acquisition unit from the keyword data stored in the keyword storage unit, in which the information search unit searches for information on the broadcast content corresponding to the state information from the content information storage unit on the basis of the keyword determined by the keyword determination unit.

(3) According to still another aspect of the present invention, in the data processing device (1) or (2), the information on the broadcast content is information on the broadcast program.

(4) According to still another aspect of the present invention, in the data processing device of (3), the information output by the output unit is a portion of the information on the broadcast program.

(5) According to still another aspect of the present invention, in the data processing device of (1), the state information acquisition unit acquires first state information indicating a state of a first body, and second state information indicating a state of a second body, and the information search unit searches for information on the broadcast content corresponding to the first state information and information on the broadcast content corresponding to the second state information.

(6) According to still another aspect of the present invention, in the data processing device of (1) or (5), the state information acquisition unit acquires a target value of the state information from the terminal device, the data processing device further includes a state analysis unit that analyzes a relationship between the state information and the target value of the state information acquired by the state information acquisition unit, and the information search unit searches for information on the broadcast content corresponding to the relationship analyzed by the state analysis unit.

(7) Another aspect of the present invention is a data processing system including a terminal device that inputs state information indicating a state of a body, and a data processing device, in which the data processing device further includes: a state information acquisition unit that acquires the state information transmitted by the terminal device; an information search unit that searches for information on broadcast content corresponding to the state information acquired by the state information acquisition unit from a content information storage unit that stores the information on the broadcast content; and an output unit that outputs information on the broadcast content searched for by the information search unit.

(8) Still another aspect of the present invention is a terminal device, including: a state information input unit that inputs state information indicating a state of a body; a communication unit that transmits the state information and receives information on broadcast content corresponding to the state information; and an information output unit that outputs information on the broadcast content received by the communication unit.

(9) According to still another aspect of the present invention, the terminal device of (8) further includes a state information measurement unit that measures at least a portion of the state information.

(10) Still another aspect of the present invention is a data processing method in a data processing device, including: a state information acquisition step of acquiring state information indicating a state of a body; an information search step of searching for information on broadcast content corresponding to the state information acquired by the state information acquisition unit from a content information storage unit that stores the information on the broadcast content; and an output step of outputting information on the broadcast content searched for in the information search step.

(11) Still another aspect of the present invention is a data processing program for causing a computer of a terminal device to execute: a state information input procedure of inputting state information indicating a state of a body; a communication procedure of transmitting the state information input in the state information input procedure and receiving information on broadcast content corresponding to the state information; and an information output procedure of outputting information on the broadcast content received in the communication procedure.

### Advantageous Effects of Invention

According to the present invention, provided information is continuously used.

### BRIEF DESCRIPTION OF DRAWINGS

FIG. 1 is a conceptual diagram illustrating an overview of a data processing system according to a first embodiment of the present invention.

FIG. 2 is a block diagram illustrating a configuration of the data processing system according to this embodiment.

FIG. 3 is a block diagram illustrating a configuration of a data processing device according to this embodiment.

FIG. 4 is a diagram illustrating an example of user registration data.

FIG. 5A is a diagram illustrating an example of input data.

FIG. 5B is a diagram illustrating an example of the input data.

FIG. 6A is a diagram illustrating an example of measurement data.

FIG. 6B is a diagram illustrating an example of the measurement data.

FIG. 6C is a diagram illustrating an example of the measurement data.

FIG. 7 is a diagram illustrating an example of keyword data.

FIG. 8 is a diagram illustrating an example of viewing data.

FIG. 9 is a diagram illustrating an example of program information.

FIG. 10 is a flowchart illustrating an example of data processing according to this embodiment.

FIG. 11 is a block diagram illustrating a configuration of a terminal device according to this embodiment.

FIG. 12A is a diagram illustrating a display example of a display unit according to this embodiment.

FIG. 12B is a diagram illustrating a display example of the display unit according to this embodiment.

FIG. 13 is a diagram illustrating another example of an advice display image.

FIG. 14 is a block diagram illustrating a configuration of a data processing device according to a second embodiment of the present invention.

FIG. 15 illustrates an example of keyword data according to this embodiment.

FIG. 16 is a flowchart illustrating an example of data processing according to this embodiment.

FIG. 17 is a block diagram illustrating a configuration of a data processing device according to a third embodiment of the present invention.

FIG. 18 illustrates an example of target data according to this embodiment.

FIG. 19A is a diagram illustrating an example of keyword data according to this embodiment.

FIG. 19B is a diagram illustrating an example of the keyword data according to this embodiment.

FIG. 20 is a flowchart illustrating an example of data processing according to this embodiment.

### DESCRIPTION OF EMBODIMENTS

### First Embodiment

Hereinafter, a first embodiment of the present invention will be described with reference to the drawings.

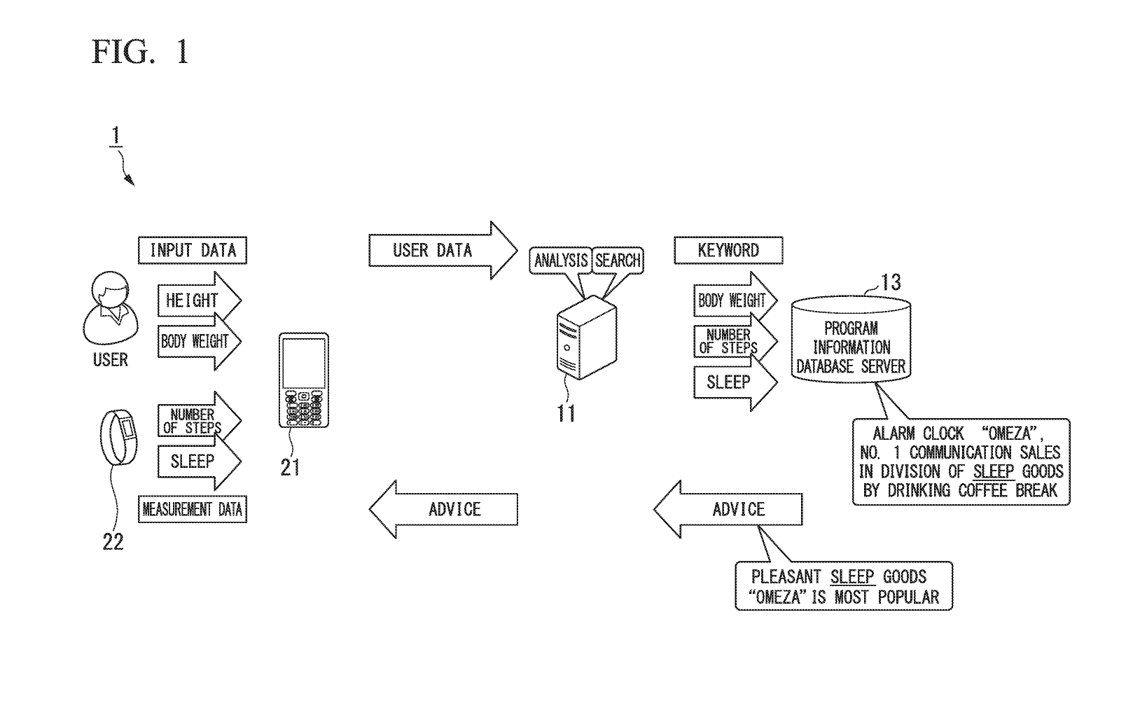

FIG. 1 is a conceptual diagram illustrating an overview of a data processing system 1 according to this embodiment.

In the data processing system 1, a terminal device 21 acquires user data indicating a state of a body of the user, and transmits the acquired user data to a data processing device 11. The user data includes input data that is input by an operation of the user, and measurement data received from a measurement device 22.

The data processing device 11 analyzes the user data received from the terminal device 21 and determines a keyword for search corresponding to the received data. In a case in which a weight, the number of steps, and a measured value of sleep are included the user data, the keywords are, for example, a body weight, the number of steps, and sleep. The data processing device 11 searches for text information including the determined keyword from program information stored in a program information database server 13 and transmits the text information that has been searched for to the terminal device 21. In the text information that has been searched for, an advice for maintaining and promoting health, such as information “pleasant sleep goods “OMEZA” is most popular”, is provided. The terminal device 21 displays the text information received from the data processing device 11.

(Configuration of Data Processing System)

Next, a configuration of the data processing system 1 according to this embodiment will be described.

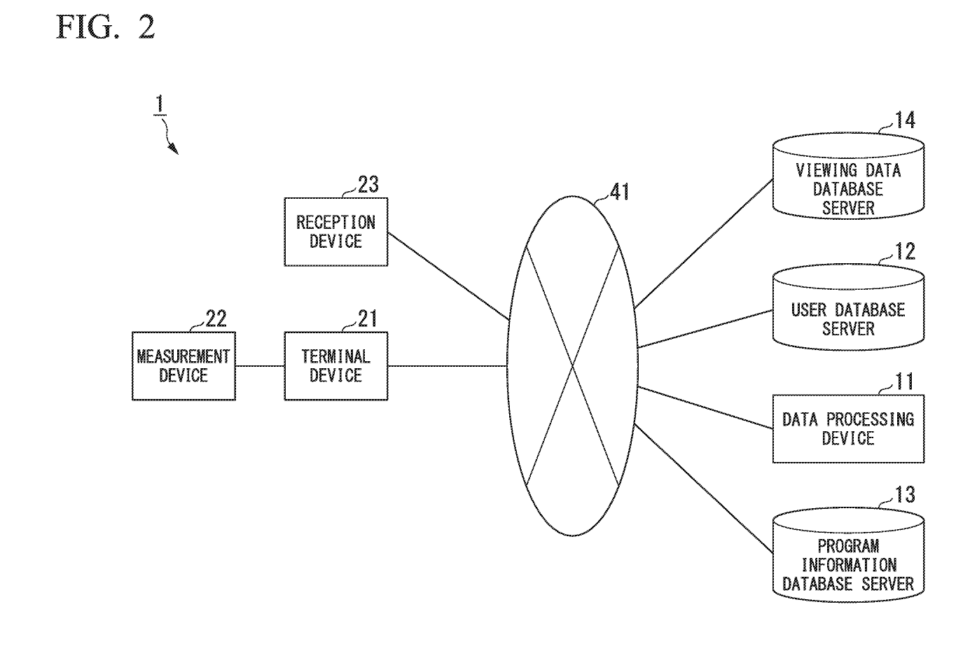

FIG. 2 is a block diagram illustrating a configuration of the data processing system 1 according to this embodiment.

The data processing system 1 includes a data processing device 11, a user database server 12, a program information database server 13, a viewing data database server 14, a terminal device 21, a measurement device 22, and a reception device 23.

The data processing device 11, the user database server 12, the program information database server 13, the viewing data database server 14, the terminal device 21, and the reception device 23 can transmit and receive data with each other over a network 41. Further, data can be transmitted and received directly between the terminal device 21 and the measurement device 22.

In the illustrated example, for simplification, the number of terminal devices 21, the measurement devices 22, or the reception devices 23 is one. The number of terminal devices 21, the measurement devices 22, or the reception devices 23 is not limited to one and may be more than one.

The data processing device 11 receives the user data indicating the state of the body with user registration data from the user database server 12 in association with each other. The data processing device 11 refers to the keyword data stored in the own device to determine a keyword corresponding to the received user data The data processing device 11 searches for the program information stored in the program information database server 13 on the basis of the determined keyword. The data processing device 11 selects the program information relating to the broadcast program that has been broadcast at a location of a predetermined user by referring to the viewing data stored in the viewing data database server 14 from the program information that has been searched for. The data processing device 11 transmits the text information included in the selected program information to the terminal device 21. The terminal device 21 that is a transmission destination is designated using the device identification information or the user registration data received from the terminal device 21.

The user database server 12 is a server device that receives the user data from the terminal device 21 and stores the received user data. User registration data is stored in the user database server 12 in advance. User identification information (user ID) for identifying an individual user and device identification information (device ID) for identifying the terminal device 21 are included in the user registration data. The user database server 12 specifies user registration data including the same device identification information as the device identification information added to the received user data, and stores the received user data and the specified user registration data in association with each other. The user database server 12 transmits the user data and the user registration data that have been newly stored, to the data processing device 11 at predetermined time intervals (for example, one day). Examples of the user data and the user registration data will be described below.

The program information database server 13 is a server device in which program information of a television program is stored. In the program information, a program name, and text information indicating an explanatory text of each corner (segment) that is a segment obtained by dividing one broadcast program are included. The program information database server 13 receives search request information from the data processing device 11. The program information database server 13 specifies text information of the corner including the search keyword indicated by the received search request information from the stored program information. The program information database server 13 transmits the specified text information of the corner to the data processing device 11. The program information database server 13 includes data obtained by a business operator viewing the program confirming content of the program and forming the content as text. The program information database server 13 may receive program information from a device (not illustrated) that the business operator has. An example of the program information will be described below.

In the following description, although a case in which a broadcast program is a television program will be described by way of example, the broadcast program may be a broadcast via a communication channel a radio program or the Internet. The broadcast via the communication channel refers to retransmission of a terrestrial program using Internet protocol (IP), as well as distribution of content via the communication channel by imitating the broadcast.

The viewing data database server 14 is a server device that receives viewing data from the reception device 23, and accumulates and stores the received viewing data as a viewing log. In the viewing data, data acquisition time information indicating a time at which the reception device 23 receives the broadcast program and displays or records the broadcast program, viewed program information indicating the received broadcast program, and region information indicating a region to which an installation position of the reception device 23 belongs are included.

The viewing data database server 14 specifies the region indicated by the program identification information received from the data processing device 11 from the stored viewed program information, and selects the viewed program information indicating the broadcast program that has been broadcast in that region. The viewing data database server 14 transmits the selected viewed program information to the data processing device 11.

The input data indicating the state of the body is input to the terminal device 21 by an operation of the user. The input data includes, for example, height, weight, gender, age, body fat, a blood glucose level, a physical amount that cannot be measured in the measurement device 22, and an index value. Further, the terminal device 21 receives measurement data indicating a state of a body of the user from the measurement device 22. The terminal device 21 transmits the received input data and the received measurement data as user data to the user database server 12 over the network 41. The terminal device 21 receives text information from the data processing device 11, and displays the received text information. The terminal device 21 is, for example, an electronic device such as a multi-function mobile phone (including a so-called smart phone) carried by a user, a tablet terminal device, or a television reception device (which is any one of a portable television reception device and a stationary television reception device). In the case in which the terminal device 21 is an electronic device (including a television reception device) having a function of receiving a television broadcast, the reception device 23 may not be used.

The measurement device 22 is a measurement device that measures an index indicating a state of the body of the user. The measurement device 22 measures any one or combination of behavior information such as the number of steps, movement distance, consumed calories, and the number of floors, and sleep time within a predetermined period of time (for example, one day). The measurement device 22 generates measurement data indicating a measured value, and transmits the generated measurement data to the terminal device 21 wirelessly or wiredly. The measurement device 22 may use, for example, a scheme conforming to near field communication standard determined by IEEE 802.15.1 in transmission and reception of data to and from the terminal device 21. The measurement device 22 may include a mounting fixture (for example, a bracelet or a necklace) for mounting on the body of the user.

The reception device 23 receives program data indicating content (video and audio) of the television broadcast program via a broadcast transmission channel (not illustrated), and presents the video, the audio, or both of the received program data. The reception device 23 selects presence or absence of reception of the program, and a broadcasting station (channel) for reception on the basis of an operation signal input through an operation of the user. The reception device 23 generates the viewing data on the basis of the received program data. Specifically, the reception device 23 specifies a time zone in which the program data is continuously received, as data acquisition time information, and specifies a program name of the program as the viewed program information. Further, the reception device 23 integrates preset device identification information (for example, TV ID) and region information (for example, zip code) into the data acquisition time information and the viewed program information that have been specified, to generate viewing data. The reception device 23 transmits the generated viewing data to the viewing data database server 14. An example of the viewing data will be described below.

The network 41 is a communication channel via which data can be transmitted and received bi-directionally. The network 41 is a wide-area wireless communication network such as the Internet or a public land mobile network (PLMN).

(Configuration of Data Processing Device)

Next, a configuration of the data processing device 11 according to this embodiment will be described.

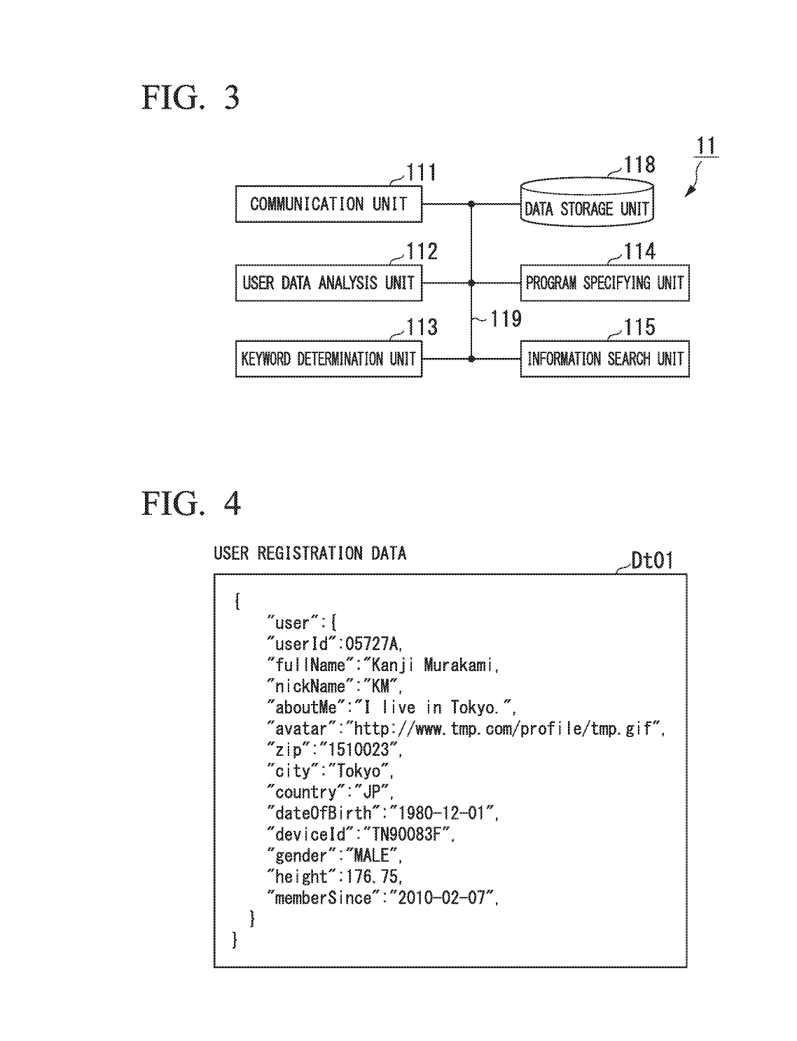

FIG. 3 is a block diagram illustrating a configuration of a data processing device 11 according to this embodiment.

The data processing device 11 includes a communication unit 111, a user data analysis unit 112, a keyword determination unit 113, a program specifying unit 114, an information search unit 115, and a data storage unit 118. The communication unit 111, the user data analysis unit 112, the keyword determination unit 113, the program specifying unit 114, the information search unit 115, and the data storage unit 118 are connected by a base line (bus) 119 which enables the units to transmit and receive data to and from each other.

The communication unit 111 transmits and receives data to and from another device. The communication unit 111 is an output unit that outputs the data received from the other device to another constituent unit, and transmits (outputs) data input from the other constituent unit to the other device. The communication unit 111 is, for example, a communication interface.

The user data analysis unit 112 receives the user data and the user registration data from the user database server 12. The user data analysis unit 112 determines element data included in the received user data. The element data is data that is an element of the user data, such as each of number-of-step data, sleep data, and the like included in the measurement data. The user data analysis unit 112 generates element data information indicating the element data determined to be included. The user data analysis unit 112 outputs the generated element data information to the keyword determination unit 113, and outputs the received user registration data to the program specifying unit 114 and the information search unit 115.

The keyword determination unit 113 refers to the keyword data stored in the data storage unit 118 to read a keyword corresponding to the element data indicated by the element data information input from the user data analysis unit 112, and determines the read keyword to be a search keyword. The keyword determination unit 113 outputs the search keyword information indicating the determined search keyword to the information search unit 115. An example of the keyword data will be described below.

The program specifying unit 114 reads the region information from the user registration data input from the user data analysis unit. The program specifying unit 114 generates program specifying information for instructing to specify a broadcast program that has been broadcast within a predetermined period of time in the region indicated by the read region information (for example, a day before that point in time). The program specifying unit 114 transmits the generated program specifying information to the viewing data database server 14 via the communication unit 111 and the network 31.

Thereafter, the program specifying unit 114 receives viewed program information indicating a broadcast program that has been broadcast in a predetermined time period in the region from the viewing data database server 14. Accordingly, the broadcast program that has been broadcast in the predetermined period in the region is specified. The program specifying unit 114 outputs the received viewed program information to the information search unit 115.

The search keyword information from the keyword determination unit 113 and the viewed program information from the program specifying unit 114 are input to the information search unit 115. The information search unit 115 generates search request information for instructing to search for text information of a corner including the search keyword indicated by the search keyword information from the broadcast programs indicated by the viewed program information. The information search unit 115 transmits the generated search request information to the program information database server 13.

Thereafter, the information search unit 115 receives the text information from the program information database server 13, and transmits the received text information to the terminal device 21. Here, the information search unit 115 specifies the terminal device 21 indicated by the device identification information included in the user registration data input from the user data analysis unit 112, as the destination.

### EXAMPLES OF VARIOUS TYPES OF DATA

Next, an example of various types of data used in this embodiment will be described.

FIG. 4 is a diagram illustrating an example of user registration data (user registration data Dt01). The user registration data Dt01 (user) includes the user identification information (userID), a name (fullName), a nickname (nickName), personal information (aboutMe), an avatar (avatar), region information (zip, city, and country), date of birth (dateOfBirth), device identification information (deviceId), gender (gender), height (height), and admission date (memberSince).

At least user identification information and device identification information may be included in the user registration data Dt01, and other information may be omitted. In a case in which the region information is omitted, the program specifying unit 114 that specifies the broadcast program that has been broadcast using the region information in the viewing data database server 14 may be omitted.

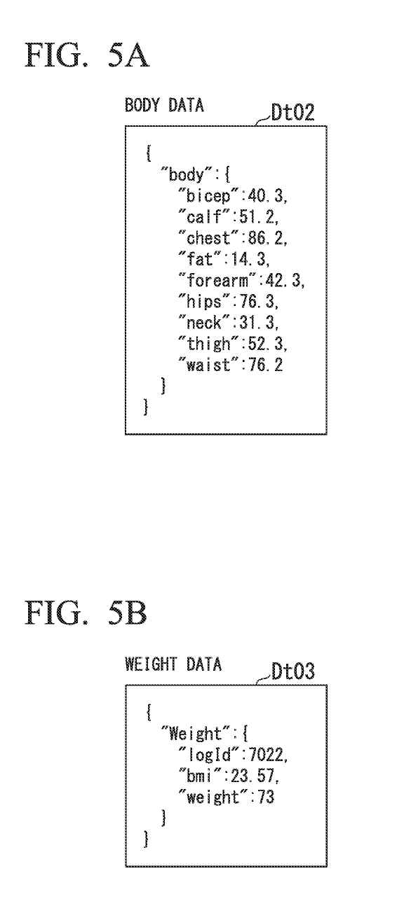

FIG. 5 is a diagram illustrating an example of the input data.

FIG. 5A illustrates an example (body data Dt02) of the body data which is a portion of the input data. The body data Dt02 (body) includes an upper arm circumference (bicep in cm), a calf circumference (calf in cm), a chest circumference (chest in cm), a body fat percentage (fat in %), a forearm circumference (forearm in cm), a hip circumference (hips in cm), a neck circumference (neck in cm), a thigh circumference (thigh in cm), and a waist circumference (waist in cm), which are associated with each other. A portion or all of the physiological data Dt02 may be omitted or may not be updated each time.

FIG. 5B illustrates an example (body weight data Dt03) of weight data which is another portion of the input data. The weight data Dt03 (Weight) includes log identification information (logId), BMI (bmi: body mass index), and a body weight (weight in kg), which are associated with each other. BMI is an index indicating a degree of obesity that is calculated from a height and a body weight. In the body weight data Dt03, BMI may be omitted.

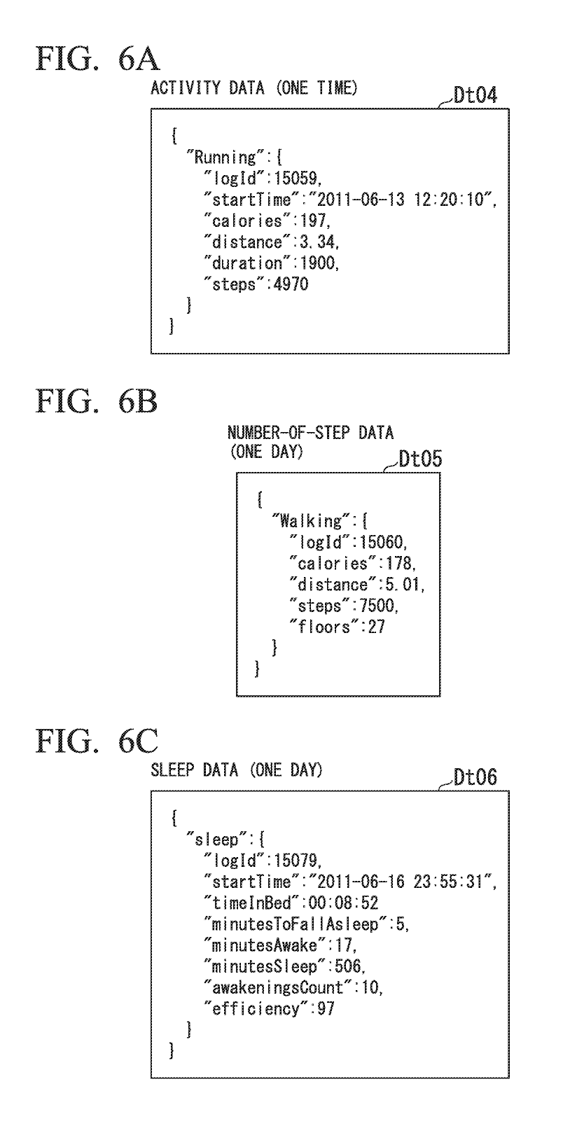

FIG. 6 is a diagram illustrating an example of measurement data.

FIG. 6A illustrates an example (activity data Dt04) of activity data that is portion of the measurement data. The activity data Dt04 (Running) is data measured during one running.

The activity data Dt04 includes log identification information (logId), start time (startTime in year-month-day hour: minute: second), consumed calories (calories in kcal), a distance (distance in km), time (duration in second), the number of steps (Steps), which are associated with each other.

In the activity data Dt04, one or both of the consumed calories and the distance may be omitted. Measurement start and end may be instructed from the measurement device 22 according to an operation of the user.

FIG. 6B illustrates an example (number-of-step data Dt05) of number-of-step data which is another portion of the measurement data. The number-of-step data Dt05 (Walking) is data measured in life of one day.

The number-of-step data Dt05 includes log identification information (logId), consumed calories (calories in kcal), a distance (in km), the number of steps, and the number of floors (in floors; the number of floors in which floors increase), which are associated with each other. In the number-of-step data Dt05, any one of the consumed calories, the distance, and the number of floors, or any combination thereof may be omitted.

FIG. 6C illustrates an example (sleep data Dt06) of sleep data which is a still another portion of the measurement data. The sleep data Dt06 (sleep) is data measured during the bedtime of one day.

The sleep data Dt06 includes log identification information (logId), start time (startTime in year-month-day hour: minute: second), bedtime (timeInBed in hour: minute: second), falling-asleep time (minutsToFallAsleep in minute), awake time (minutsAwake in minute), sleep time (minutesSleep in minute), awakening count (awakeningsCount in number of times), and sleep efficiency (efficiency in %), which are associated with each other.

In the sleep data Dt06, any one or any combination of the start time, the bedtime, the falling-asleep time, the awake time, and the awakening count (and sleep efficiency (efficiency in %) may be omitted.

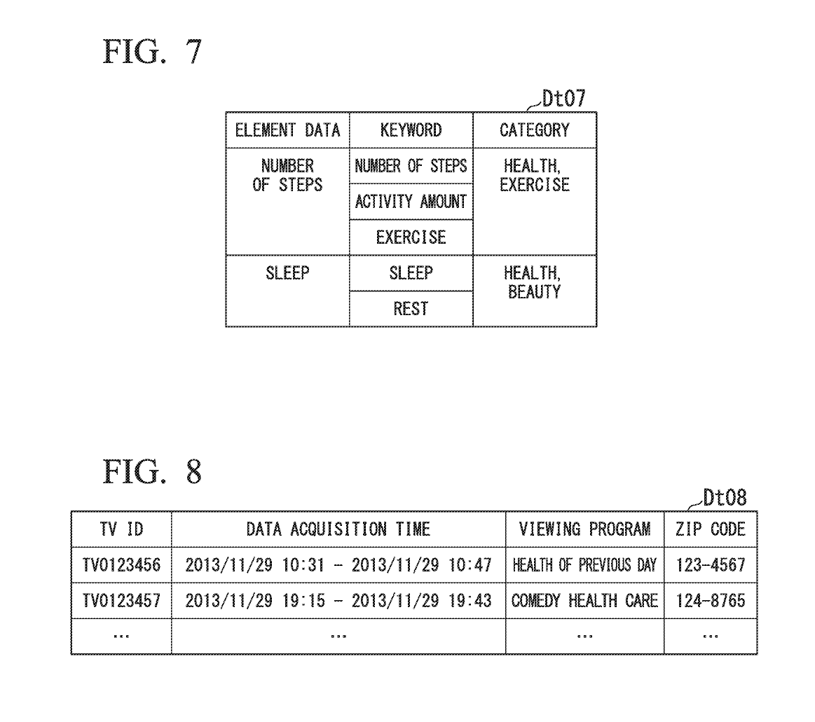

FIG. 7 is a diagram illustrating an example (keyword data Dt07) of the keyword data.

The keyword data Dt07 includes element data, a keyword, and category information, which are associated with each other. As the keyword, a topic regarding the element data and a phrase frequently included in the topic are used. The topic includes means that helps maintenance and promotion of health, such as product, service, habit, exercise, or food. The category information indicates a field and a category to which the topic regarding the element data belongs.

Further, the keyword data Dt07 is used in a case in which the input user data includes one or both of the number-of-step data and the sleep data as element data. In the keyword data Dt07, a plurality of keywords may be associated with one item of element data or a plurality of categories may be associated with one item of element data.

For example, in a second row of FIG. 7, for example, three keywords “number of steps”, “activity amount”, and “exercise”, and two categories “health”, and “exercise” are associated with “number of steps” as the element data.

Here, a process of the keyword determination unit 113 will be described on the assumption that the element data information input from the user data analysis unit 112 indicates “number of steps” data.

The keyword determination unit 113 refers to the keyword data Dt07 to read keywords “number of steps”, “amount of activity”, and “exercise” corresponding to the data “number of steps”. The keyword determination unit 113 outputs search keyword information indicating the read keywords “number of steps”, “amount of activity”, and “exercise” to the information search unit 115.

Further, the keyword determination unit 113 refers to the keyword data Dt07 to read the category “health”, and “exercise” corresponding to the number-of-step data. The keyword determination unit 113 outputs search category information indicating the read categories “health” and “exercise” to the information search unit 115.

The information search unit 115 generates search request information for instructing to search for text information of a corner including at least one of the keywords “number of steps”, “amount of activity”, and “exercise” indicated by the search keyword information (OR search) and belonging to at least one of the categories “health” and “exercise” indicated by the search category information (OR search). Therefore, the text information that is searched using the keyword indicated by the search keyword information is limited to the category indicated by the search category information.

FIG. 8 is a diagram illustrating an example (viewing data Dt08) of viewing data.

The viewing data Dt08 includes device identification information (TV ID), data acquisition time information, viewed program information, and region information (zip code), which are associated with each other. The device identification information is information for identifying the reception device 23 viewed by the user. The data acquisition time information is information indicating a time zone in which one item of program data is continuously received, that is, information indicating a viewing time of the program. The data acquisition time information is designated using a start time and an end time. The viewed program information is information indicating a program name of the program. The region information includes information indicating a region in which the reception device 23 is installed. In the illustrated example, the region information is a zip code. In a second row of FIG. 8, the data acquisition time (start time 2013/11/29 10:31-end time 2013/11/29 19:43), the viewed program information “health of yesterday”, and region information “123-4567” are associated with the device identification information (TV0123456).

As described above, the viewing data Dt08 is used when the program specifying unit 114 narrows down a broadcast program that is a search target using a broadcasting time and a broadcasting region. For example, the program specifying unit 114 can refer to a second row of the viewing data Dt08 to specify a program name “health of yesterday” of the broadcast program in which a predetermined time zone includes data acquisition time 2013/11/29 10:31-2013/11/29 10:47, and which has been broadcast in a region 123-4567 matching a region indicated by a zip code indicated by the user registration information. Accordingly, the text information that is a search target is limited by a broadcasting time and a broadcast region.

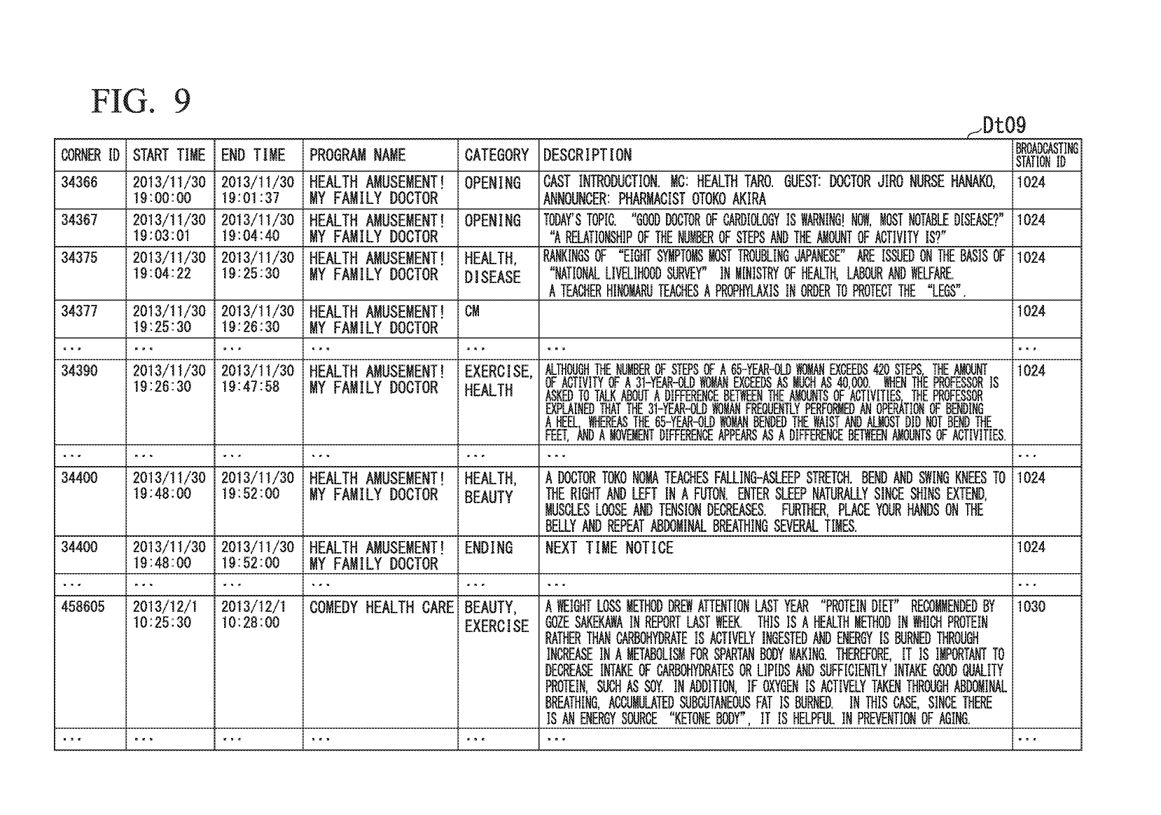

FIG. 9 is a diagram illustrating an example (program information Dt09) of the program information.

The program information Dt09 includes corner identification information (corner ID), start time information, end time information, program name information, category information, text information (description), and broadcasting station identification information (broadcasting station ID), which are associated with each other. The corner identification information is information for identifying each corner, and is uniquely associated with each corner. The start time information is information indicating a start time of the corner. The end time information is information indicating an end time of the corner. The program name information is information indicating a name of a broadcast program (program name) including the corner. The category information is information indicating a field and a category to which the program belongs. The text information is information indicating explanatory text of a thing broadcast in the corner. The text information may include a topic relating to health, such as a thing regarding maintenance and promotion of health. For example, an overview of the corner, speech information indicating speech of a performer, proceedings information, a commentary article, and the like may be included.

For example, in a ninth row of FIG. 9, start time information “2013/11/30 19:48:00”, end time information “2013/11/30 19:52:00”, program name information “health amusement! My family doctor”, category information “health, beauty”, and text information (description) “A doctor TOKO NOMA teaches falling-asleep stretch. Bend the knee in the futon . . . place your hands on the belly and repeat abdominal breathing many times.”, and broadcasting station identification information “1024” are associated with the corner identification information “34400”.

The text information is a primary search target in the information search unit 115. Further, the category information may be used when the text information that has been searched for is restricted.

Although a case in which the text information is a primary search target in the information search unit 115 will be described by way of example for convenience of description, the search target may be, for example, all of a row of FIG. 9 or may be a portion thereof. For example, in a case in which the text information is the search target and the text information is displayed as an advice on the terminal device 21, the information search unit 115 may extract start time information, program name information, or the like corresponding to the text information together. The terminal device 21 may display the start time information, the program name information, or the like that has been extracted in an advice display image (which will be described below).

(Data Processing)

Next, an example of data processing according to this embodiment will be described.

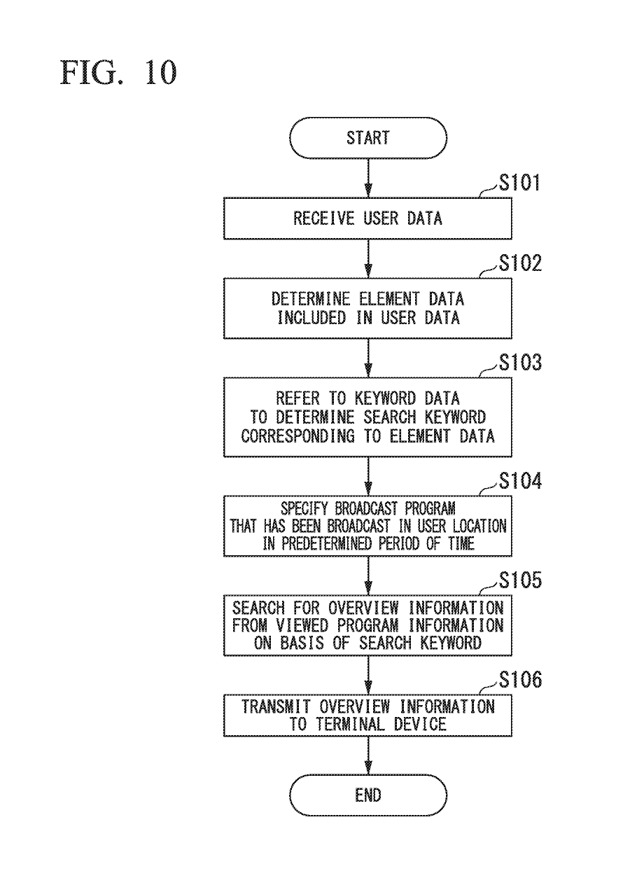

FIG. 10 is a flowchart illustrating an example of data processing according to this embodiment.

(Step S101) The user data analysis unit 112 of the data processing device 11 receives the user data and the user registration data from the user database server 12. The process then proceeds to step S102.

(Step S102) The user data analysis unit 112 determines the element data included in the received user data. The process then proceeds to step S103.

(Step S103) the keyword determination unit 113 refers to the keyword data stored in the data storage unit 118 to read the keyword corresponding to each item of element data determined to be included, and determines the read keyword to be a search keyword. The process then proceeds to step S104.

(Step S104) The program specifying unit 114 generates program specifying information for instructing the viewing data database server 14 to specify a broadcast program that has been broadcast in a predetermined period of time (for example, a day before that point in time) in the region (location of the user) indicated by the region information included in the user registration data. Thereafter, the program specifying unit 114 receives the viewed program information indicating the broadcast program that has been broadcast in a predetermined time in the region from the viewing data database server 14. The process then proceeds to step S105.

(Step S105) The information search unit 115 instructs the program information database server 13 to search for text information of the corner including the search keyword indicated by the search keyword information in the broadcast program indicated by the received viewed program information. Then, the information search unit 115 receives the text information from the program information database server 13. The process then proceeds to step S106.

(Step S106) The information search unit 115 specifies the terminal device 21 indicated by the device identification information included in the user registration data as a transmission destination, and transmits the received text information to the terminal device 21. The process illustrated in FIG. 10 then ends.

(Configuration of the Terminal Device)

Next, a configuration of the terminal device 21 will be described.

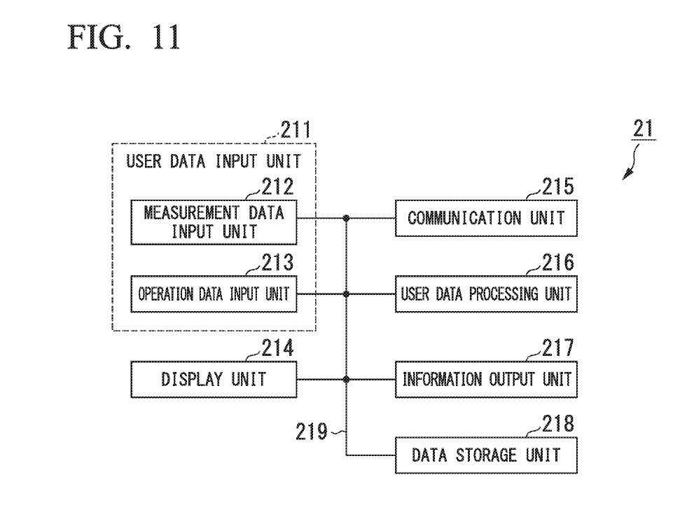

FIG. 11 is a block diagram illustrating a configuration of the terminal device 21 according to this embodiment.

The terminal device 21 includes a user data input unit 211, a display unit 214, a communication unit 215, a user data processing unit 216, an information output unit 217, and a data storage unit 218. The user data input unit 211 includes a measurement data input unit 212 and an operation data input unit 213. The measurement data input unit 212, the operation data input unit 213, the display unit 214, the communication unit 215, the user data processing unit 216, the information output unit 217, and the data storage unit 218 are connected using a base line (bus) 219 which enables the units to transmit and receive data to and from each other.

The measurement data from the measurement device 22 is input to the measurement data input unit 212, and the input measurement data is output to the user data processing unit 216. The measurement data input unit 212 is, for example, a wireless communication interface.

The operation data input unit 213 generates various operation input signals according to an operation of a user, and outputs the generated operation input signal to the respective units. The operation input signal includes input data indicating a state of a body of the user. The operation data input unit 213 outputs the input data to the user data processing unit 216. The operation data input unit 213 is, for example, a pointing device such as a touch sensor, a keyboard, or a mouse disposed to overlap the display unit 214.

The display unit 214 displays an image based on image data input from the information output unit 217. The display unit 214 is, for example, a liquified crystal display (LCD).

The communication unit 215 transmits and receives data to and from other devices. The communication unit 215 transmits the user data input from the user data processing unit 216 to the user database server 12. Further, the communication unit 215 outputs text information received from the data processing device 11 to the information output unit 217. The communication unit 215 is, for example, a communication interface.

The user data processing unit 216 converts the measurement data input from the measurement data input unit 212 and the operation data input from the operation data input unit 213 into respective predetermined formats (see FIGS. 5A, 5B, 6A, 6B, and 6C). The user data processing unit 216 transmits the data obtained by converting the format as user data to the user database server 12 via the communication unit 215.

The information output unit 217 receives the text information from the data processing device 11. The information output unit 217 converts the received text information into image data of a predetermined format, and outputs the converted image data to the display unit 214. Thus, the received text information is displayed as an advice display image in the display unit 214.

The data storage unit 218 stores data used for the process that is performed by the terminal device 21 or data generated through the process. An incoming call display image format is stored in the data storage unit 218 in advance. The incoming call display image format is information indicating various characters, buttons, and sizes or arrangements thereof that are displayed in the incoming call display image (which will be described below).

(Example of Display)

Next, a display example of the display unit 214 according to this embodiment will be described.

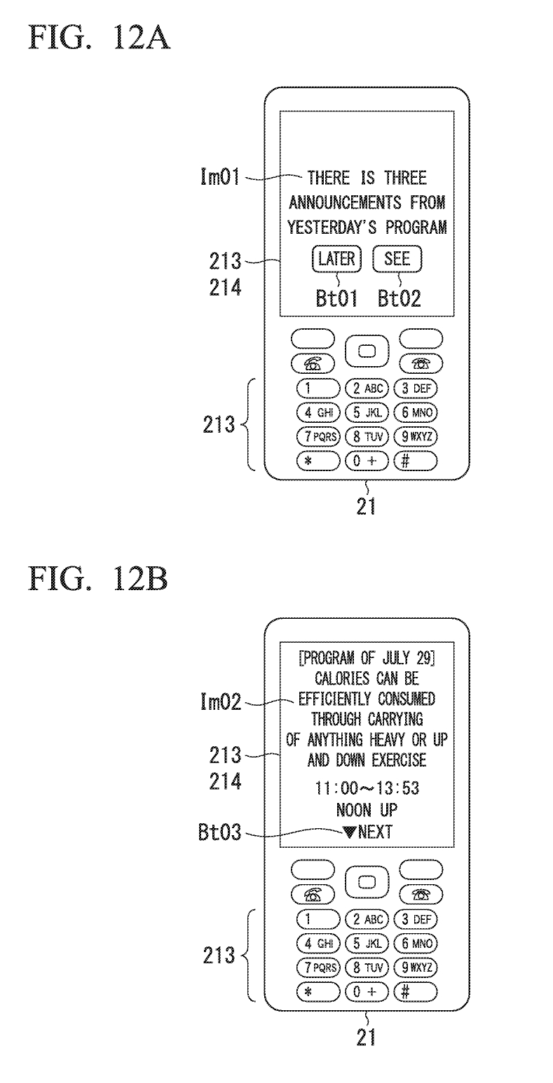

FIGS. 12A and 12B are diagrams illustrating a display example of the display unit 214 according to this embodiment. FIGS. 12A and 12B illustrate a display example in a case in which the terminal device 21 is a multi-functional mobile phone. The terminal device 21 has a vertically long flat shape in which a length in a vertical direction is larger than a length in a horizontal length, and includes a display unit 214 on a surface thereof. The terminal device 21 includes a touch sensor at a position overlapping a surface of the display unit 214 as the operation data input unit 213, and includes a keyboard at a position not overlapping the display unit 214.

FIG. 12A illustrates an example (incoming call display image Im01) of an incoming call display image. The incoming call display image Im01 is an image for notifying a user that an advice extracted from the broadcast program is incoming. The incoming call display image Im01 includes text information “there is three announcements from yesterday's program” indicating the fact and the number of cases, and two buttons Bt01 and Bt02. The button Bt01 is a button for rejecting a display of the advice. In the button BT01, a character string “Later” indicating that the display is rejected is displayed. The button Bt02 is a button for instructing a display of the advice display image. In the button Bt02, a character string “see” indicating that the display is instructed is displayed.

When the information output unit 217 displays the incoming call display image, the information output unit 217 reads an incoming call display image format from the data storage unit 218 and counts the number of cases of received text information. The information output unit 217 integrates the counted number of cases into the read incoming call display image format to generate incoming call display image data, and outputs the generated incoming call display image data to the display unit 214. When an operation input signal input from the operation data input unit 213 indicates pressing of the button BT01, the information output unit 217 erases the incoming call display image displayed in the display unit 214. Further, when the operation input signal input from the operation data input unit 213 indicates pressing of the button Bt02, the information output unit 217 converts a predetermined portion (for example, a first corner) of the received text information and the other element of the broadcast program information corresponding to the portion, and outputs a result thereof to the display unit 214. Thus, an advice display image is displayed the display unit 214.

FIG. 12B illustrates an example (advice display image Im02) of the advice display image. In the advice display image Im02, a broadcasting day of a program “program of July 29” according to the received text information, text information “calories can be efficiently consumed through carrying of anything heavy or up and down exercise.”, a broadcasting time “11:00 to 13:53”, a program name “noon up”, and a button Bt03 are displayed in order from top to bottom. The button Bt03 is a button for instructing a display of the text information of a next order. The broadcasting date and the broadcasting time are specified from the start time information and the end time information of the broadcast program information, and the program name is specified from the program name information of the broadcast program information.

When the operation input signal input from the operation data input unit 213 indicates pressing of the button Bt03, the information output unit 217 specifies the text information of the next corner of the currently displayed text information. The information output unit 217 converts the specified text information and another element of the broadcast program information corresponding to a portion thereof and outputs a result thereof to the display unit 214. Thus, an advice display image including text information of the next corner is displayed in the display unit 214.

A broadcasting station name may be included in the advice display image, and a broadcasting time of the corner may be included in place of the broadcasting time of the program. Thus, the advice display image, the user, a leading information broadcast by when and how the timing of which program can be strong impression that. Since the information included in the broadcast content may be considered to be highly credible in comparison with hearsay or the like, the text information displayed as the advice can be recognized as valid.

In the text information displayed as an advice, content may be the same as or may be different from a daily behavior of the user. Any of the information is information useful for the user. For example, the user can confirm that an action of the user is correct by referring to information indicating similar or the same content to or as that of the action performed by the user, which encourages a more sustained action. In addition, the user learns a new method by referring to information indicating different content from that of the action performed by the user.

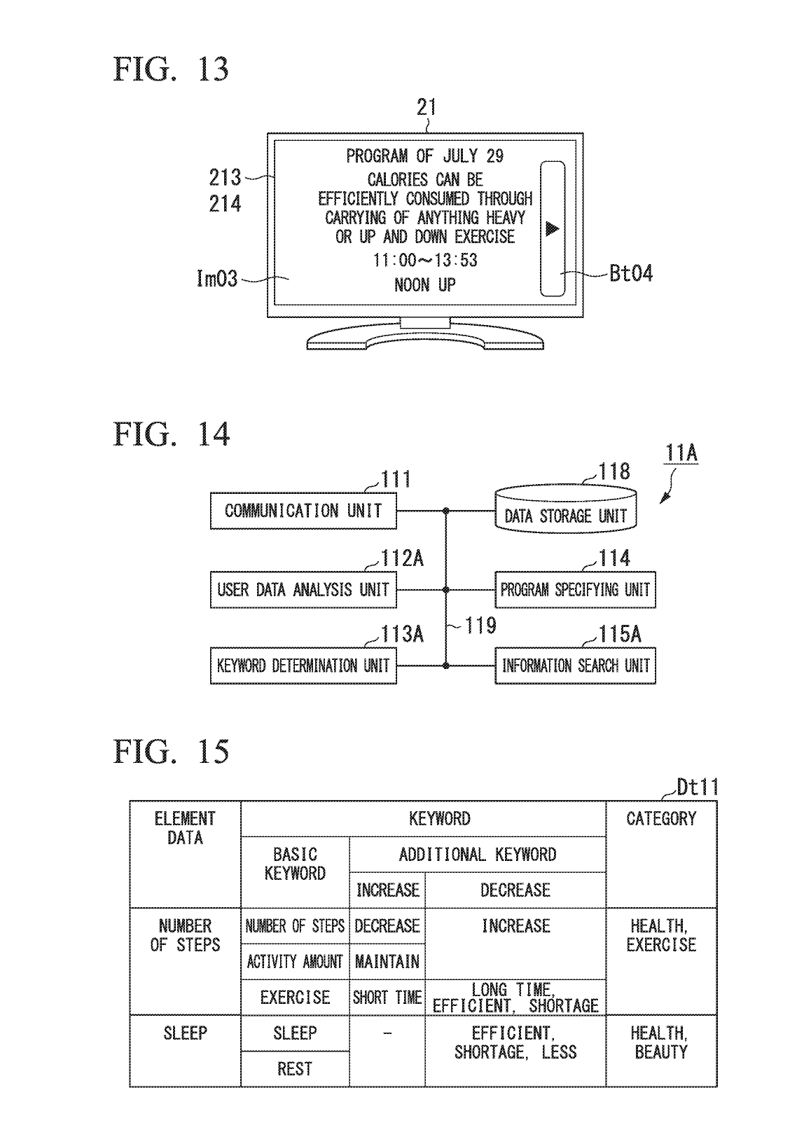

FIG. 13 is a diagram illustrating another example (advice display image Im03) of the advice display image.

The example illustrated in FIG. 13 is a display example in a case in which the terminal device 21 is a television reception device. The terminal device 21 has a vertical elongated shape shorter towards the vertical length than the horizontal length, a display unit 214 to substantially the entire surface thereof. A touch sensor as the operation data input unit 213 overlaps a display unit 214. In this example, the same text information as text information used for a display of the advice display image Im02 is used.

Broadcast date, text information, broadcasting time, and a program name in information displayed on the advice display image Im03 are the same as in the advice display image IM02. However, the advice display image Im03 is different from the advice display image Im02 including the button Bt03 near the bottom side in that a button Bt04 is displayed near a right side. Further, a process of the information output unit 217 when the button Bt04 is pressed is the same as the process when the button Bt04 is pressed.

As illustrated in FIG. 13, in a case in which a display area is larger than that of the mobile phone, the terminal device 21 may omit a display (FIG. 12A) of the incoming call display image. Further, the terminal device 21 may include another video or text data in the display image (any one or both of the incoming call display image and the advice display image). Further, the terminal device 21 may report content of the display image by voice instead of or together the display of the display image.

As described above, the data processing device 11 according to this embodiment includes the keyword storage unit (for example, the data storage unit 118) that stores the keyword data including the keywords regarding the state information indicating the state of the body. The data processing device 11 includes the state information acquisition unit (for example, the user data analysis unit 112) that acquires the state information indicating the state of the body transmitted by the terminal device (for example, the terminal device 21). Further, the data processing device 11 includes the keyword determination unit (for example, keyword determination unit 113) that determines the keyword corresponding to the state information acquired by the state information acquisition unit by referring to the keyword data. Further, the data processing device 11 includes the information search unit (for example, information search unit 115) that searches for text information indicating explanatory text regarding state information from the content information storage unit (for example, program information database server 13) that stores text information regarding content on the basis of the keyword determined by the keyword determination unit. A content information unit that stores text information regarding content is not limited to the program information database server 13, and may be a server device connected to the network 41. The information search unit 115 may attempt to search for text information from another server device connected to the network 41 in a case in which search information of the text information based on the keyword is not obtained in one server device.

Further, the terminal device 21 according to this embodiment includes a state information input unit (for example, user data input unit 211) that inputs state information indicating the state of the body. Further, the terminal device 21 includes a communication unit (for example, communication unit 215) that transmits the state information to a data processing device (for example, data processing device 11) or a device (for example, user database server 12) that transmits the state information to the data processing device, and receives the text information searched for by the data processing device. Further, the terminal device 21 includes an information output unit (for example, information output unit 217) that outputs the text information received by the communication unit.

With this configuration, the text information including a phrase regarding the state of the body indicated by the state information received from the terminal device 21 as a keyword is searched for. Since the text information corresponding to the state of the body of the user is acquired as the text information that has been searched for, the acquired text information is continuously used without being bored of the user. Further, since the acquired text information relates to existing content, the content is effectively utilized as resources. Further, the content may be a broadcast program. In this case, a new broadcast program is always stored by a producer or a broadcaster. With this configuration, the text information corresponding to the new broadcast program as the search text information is acquired, and the acquired text information is continuously used without being bored of the user.

### Second Embodiment

Next, a second embodiment of the present invention will be described. The same configurations as in the above-described embodiment are denoted with the same reference signs and are incorporated by reference.

A data processing system 1A (not illustrated) according to this embodiment includes a data processing device 11A in place of the data processing device 11 in the data processing system 1 (FIG. 2).

FIG. 14 is a block diagram illustrating a configuration of the data processing device 11A according to this embodiment.

The data processing device 11A includes a communication unit 111, a user data analysis unit 112A, a keyword determination unit 113A, a program specifying unit 114, an information search unit 115, and a data storage unit 118.

The user data analysis unit 112A generates the element data information indicating the element data included in the user data received from the user database server 12 and outputs the generated element data information to the keyword determination unit 113, similar to the user data analysis unit 112. Further, the user data analysis unit 112A outputs the received user registration data to the program specifying unit 114 and the information search unit 115.

The user data analysis unit 112A analyzes the change trend over time with respect to the data indicating a quantitative value in the element data determined to be included in the received user data. In the analysis, the value of the element data from the point in time to a predetermined previous time (for example, for one week) is subjected to regression analysis to the linear function. The user data analysis unit 112A, in a case in which the slope of the linear function obtained through analysis is greater than threshold value 1, the user data analysis unit 112A determines that the value of the element data increases. Threshold value 1 is a predetermined positive threshold value. Further, in a case in which the slope is smaller than threshold value 2, the user data analysis unit 112A determines that the value of the element data decreases. Threshold 2 is a predetermined negative threshold value. Further, in a case in which the slope is equal to or greater than threshold value 2 and smaller than or equal to threshold value 1, the user data analysis unit 112A determines that the value of the element data is unchanged. The user data analysis unit 112A generates element data change information indicating whether the value of the element data increases, decreases, or is unchanged, and outputs the generated element data change information to the keyword determination unit 113.

The keyword determination unit 113A determines a keyword corresponding to the element data, similar to the keyword determination unit 113. Further, the keyword determination unit 113A determines a category corresponding to the element data and outputs search category information indicating the determined category to the information search unit 115A. However, in this embodiment, an additional keyword is further included in the keyword data stored in the data storage unit 118. The additional keyword is associated with each of the change trend (increase, decrease, and unchanged) of the element data. In the following description, the keyword described in the first embodiment is referred to as a base keyword and is distinguished from the additional keyword. The additional keyword included in the keyword data is also associated with the base keyword corresponding to the element data. An example of keyword data according to this embodiment will be described below.

The keyword determination unit 113A refers to the keyword data to read additional keyword corresponding to the change trend of the element data indicated by the element data change information input from the user data analysis unit 112A. The keyword determination unit 113A forms a keyword set that is a set of read additional keyword and the basic keyword associated with the additional keyword, and outputs the search keyword information indicating the formed keyword set to the information search unit 115A.

The search keyword information from the keyword determination unit 113A is input to the information search unit 115A, and the viewed program information from the program specifying unit 114 is input to the information search unit 115A.

The information search unit 115A generates search request information for instructing to search for text information of the corner including the basic keyword and the additional keyword corresponding to the basic keyword in the keyword set indicated by the search keyword information in the broadcast program indicated by the viewed program information. The information search unit 115A transmits the generated search request information to the program information database server 13.

Thereafter, the information search unit 115A transmits the received text information to the terminal device 21, similar to the information search unit 115.

(Example of Keyword Data)

Next, an example of keyword data according to this embodiment will be described.

FIG. 15 illustrates an example (keyword data Dt11) of the keyword data according to this embodiment.

The keyword data Dt11 includes element data, a keyword, and category information, which are associated with each other, similar to the keyword data Dt07 (FIG. 7). In the keyword data Dt11, the keyword includes a base keyword and an additional keyword, which are associated with each other. The additional keyword is further determined for each change trend of the element data. In the following description, the additional keyword determined for each change trend of the element data is referred to as a trend-specific additional keyword. Further, this is a combination of the basic keyword and the corresponding trend-specific additional keyword

In the keyword data Dt11, an additional keyword may be associated with each of the plurality of keywords associated with one item of element data.

For example, in a second row of FIG. 15, for example, three basic keywords “number of steps”, “activity amount”, and “exercise” are associated with the “number of steps” data as the element data. Additional keywords are associated to the respective basic keywords “number of steps”, “active amount”, and “exercise”. “decrease” and “increase” as additional keywords are associated with the basic keyword “number of steps”. The additional keyword “decrease” is a trend-specific additional keyword corresponding to “increase” as a change trend of the element data “number of steps”. The additional keyword “increase” is a trend-specific additional keyword corresponding to “decrease” as a change trend of the element data “number of steps”. Thus, as the trend-specific additional keyword, a phrase indicating a change trend of a thing indicated by the corresponding basic keyword, and a phrase regarding maintenance and improvement of health according to the change trend of the element data may be used. Further, the trend-specific additional keyword may be a topic relating to a change trend of the corresponding element data, the phrase regarding maintenance and improvement of health according to the change trend of the element data, or a phrase frequently included in the topic.

Further, a plurality of trend-specific additional keywords may be determined in the change trend of one item of element data. For example, three trend-specific additional keywords “long time”, “efficient”, and “shortage” are associated with the basic keyword “exercise” according to the change trend “decrease” of the element data “number of steps” in a second line of FIG. 15. Further, in the change trend of each item of element data, the trend-specific additional keyword may not be necessarily determined. For example, the trend-specific additional keyword corresponding to the change trend “increase” of the element data “sleep” in a third row of FIG. 15 is not determined. The trend-specific additional keyword corresponding to the change trend “unchanged” is not determined for both of the element data “number of steps” and “sleep”. This is because, in a case in which the change trend of the element data indicates maintenance and improvement of health as described above, it is not using necessarily necessary to search for text information according to the change trend using the trend-specific additional keyword.

Here, the process of the keyword determination unit 113A will be described in an example in which the element data information input from the user data analysis unit 112A is “sleep” and the element data change information indicates “reduction”.

The keyword determination unit 113A refers to the keyword data Dt11 to read basic keywords “sleep” and “rest” corresponding to the element data information “sleep”. The keyword determination unit 113A reads trend-specific additional keywords “efficient”, “shortage” and “less” corresponding to the element data information “sleep” and the change tend “decrease” indicated by the element data change information. The trend-specific additional keywords “efficient”, “shortage” and “less” are parallel with one another.

The keyword determination unit 113A forms keyword sets “sleep-efficiency”, “sleep-shortage”, “sleep-less”, “rest-efficiency”, “rest-shortage”, and “rest-less” that are sets of each of read base keywords “sleep” and “rest” and each of the trend-specific additional keywords “efficient”, “shortage” and “less” corresponding thereto. The keyword determination unit 113A outputs the search keyword information indicating each of the formed keyword sets to the information search unit 115A.

Further, the keyword determination unit 113A refers to the keyword data Dt11 to read category information “health” and “beauty” corresponding to the element data information “sleep”, and outputs the read category information “health” and “beauty” as the category information to the information search unit 115A.

The information search unit 115A generates search request information including the basic keyword “sleep” and the trend-specific additional keyword “efficient” that form the keyword set “sleep-efficiency” indicated by the search keyword information (AND search) with respect to the keyword set (OR search) and instructing to search for text information of the corner belonging to at least one of the categories “health” and “beauty” indicated by the search category information (OR search). Therefore, a sum set between keyword sets of text information of the corner including the basic keyword and the corresponding trend-specific additional keyword that form each keyword set is searched for. The sum set that has been searched for is limited by the category indicated by the search category information.

In a case in which the trend-specific additional keyword corresponding to the change trend of the element data is not determined in the keyword data, the keyword determination unit 113A generates search keyword information indicating the basic keyword corresponding to the element data, similar to the keyword determination unit 113. For example, in the example illustrated in FIG. 15, the trend-specific additional keyword corresponding to the element data “sleep” and “the element data change information “increase” is not determined. In this case, the keyword determination unit 113A generates the search keyword information indicating basic keywords “sleep” and “rest” corresponding to the element data information “sleep”, and outputs the generated search keyword information to the information search unit 115A. The information search unit 115A generates the search request information including at least one of the keywords “sleep” and “rest” indicated by the input search keyword information (OR search) and instructing to search for text information of a corner belonging to at least one of categories “health” and “beauty” (OR search) indicated by the search category information, similar to the information search unit 115.

(Data Processing)

Next, an example of data processing according to this embodiment will be described.

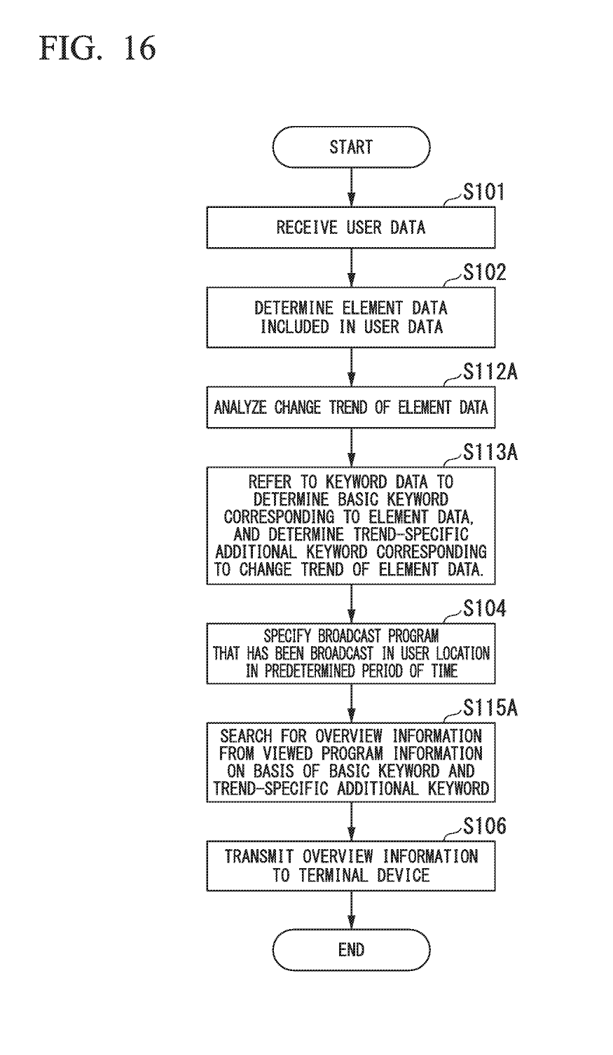

FIG. 16 is a flowchart illustrating an example of data processing according to this embodiment.

The data processing according to this embodiment includes steps S101, S102, S112A, S113A, S104, S115A, and S106.

In this embodiment, the user data analysis unit 112A executes a process of step S102, and the information search unit 115A executes a process of step S106.

In the data process illustrated in FIG. 16, after the process of steps S101 and S102 ends, the process proceeds to step S112A.

(Step S112A) The user data analysis unit 112A analyzes a change trend over time of the value of the element data included in the received user data, and determines whether the value of the element data increases, decreases, or un-changes. The process then proceeds to step S113A.

(Step S113A) The keyword determination unit 113A refers to the keyword data to determine a basic keyword corresponding to the element data, and determine a trend-specific additional keyword corresponding to the change trend of the element data. The keyword determination unit 113A forms a keyword set of the basic keywords and the additional keyword that have been determined. Further, the keyword determination unit 113A determines a category corresponding to the element data. The process then proceeds to step S104. After the process of step S104 ends, the process proceeds to step S115A.

(Step S115A) The information search unit 115A instructs the program information database server 13 to search for text information of the corner including the basic keyword and the additional keyword corresponding to the basic keyword in the keyword set determined by the keyword determination unit 113A in the broadcast program specified by the program specifying unit. Thereafter, the information search unit 115A receives the text information from the program information database server 13. The process then proceeds to step S106. After the process of step S106 ends, the process illustrated in FIG. 16 ends.

As described above, the data processing device 11A according to this embodiment includes the state analysis unit (for example, user data analysis unit 112A) that analyzes a change in the acquired state information. Further, in the data processing device 11A, the keyword data stored in the keyword storage unit (for example, data storage unit 118) includes a second keyword (for example, additional keyword) corresponding to a change in state information. Further, in the data processing device 11A, the keyword determination unit (for example, keyword determination unit 113A) further selects the second keyword corresponding to a change in the state information analyzed by the state analysis unit.

With this configuration, the text information including the phrase regarding the state of the body indicated by the state information received from the terminal device 21 and the phrase regarding the change in state of the body as keywords is searched for. As the text information that has been searched for, text information suitable for the change in state of the body of the user is acquired, and text information having a low relevance to the change in state of the body of the user is excluded. Therefore, it is possible to improve likelihood of desired information regarding health according to changes in state of the body being acquired.

### Third Embodiment

Next, a third embodiment of the present invention will be described. The same configurations as in the above-described embodiment are denoted with the same reference signs and incorporated by reference.

A data processing system 1B (not illustrated) according to this embodiment includes a data processing device 11B in place of the data processing device 11 in the data processing system 1 (FIG. 2).

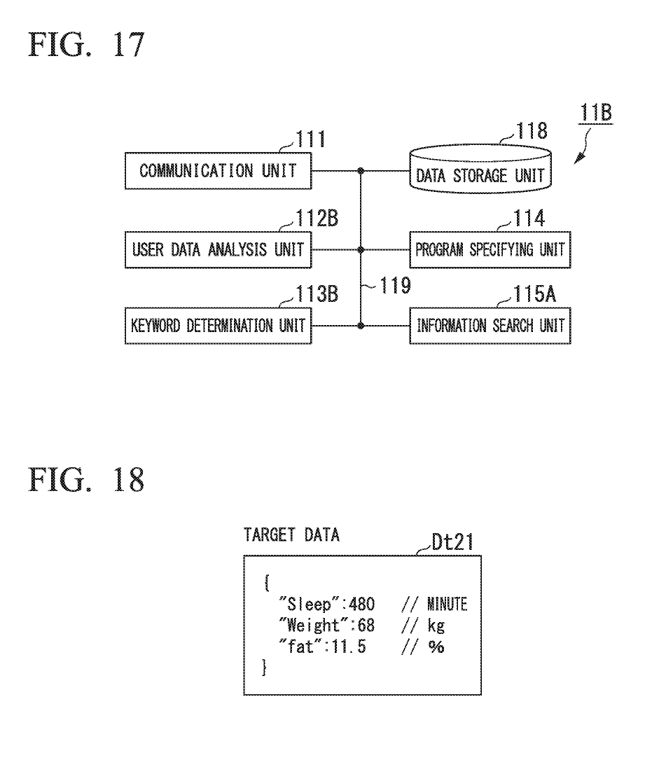

FIG. 17 is a block diagram illustrating a configuration of the data processing device 11B according to this embodiment.

The data processing device 11B includes a communication unit 111, a user data analysis unit 112B, a keyword determination unit 113B, a program specifying unit 114, an information search unit 115A, and a data storage unit 118.

The user data analysis unit 112B generates the element data information indicating the element data included in the user data received from the user database server 12 and outputs the generated element data information to the keyword determination unit 113, similar to the user data analysis unit 112. Further, the user data analysis unit 112B outputs the received user registration data to the program specifying unit 114 and the information search unit 115A.

A target value of the element data is set in the user data analysis unit 112B. The user data analysis unit 112B may directly receive target data including the target value input by an operation of the user from the terminal device 21 or may receive the target data via the user database server 12.

The user data analysis unit 112B determines whether the element data determined to be included in the received user data, which is data indicating a quantitative value, reaches the set target value. When a difference value between the value of the element data and the target value is in a range between threshold value 3 (lower limit) and threshold value 4 (upper limit), the user data analysis unit 112B determines that the value of the element data reaches the target value (achievement). Threshold 3 is a predetermined threshold value of a positive difference value. Threshold 4 is a predetermined threshold value of a negative difference value. When an absolute value of a difference between the value of the element data and the target value is smaller than threshold value 3 or greater than threshold value 4, the user data analysis unit 112B determines that the value of the element data does not reach the target value (non-achievement).

According to a type of element data, any one of threshold value 3 and threshold value 4 may be omitted. That is, in a case in which the value of the element data is equal to the target value (threshold value) or is a value indicating a state more preferable than the target value, the user data analysis unit 112B may determine that the value of the element data reaches the target value. For example, in a case in which the element data is a body weight, the user data analysis unit 112B determines that the value of the element data reaches the target value when the value of the element data is equal to or smaller than the target value. Such a determination scheme may be applied to element data such as a cholesterol value or blood pressure other than the body weight.

The user data analysis unit 112B further analyzes whether the value of the element data is away from the target value, approaches the target value, or is unchanged as a change trend over time of the value of the element data with respect to the target value. For example, in a case in which threshold values 3 and 4 are set, the user data analysis unit 112B performs regression analysis of a difference value between the value of the element data and the target value from the point in time to lapse of a predetermined time (for example, for one week) to a linear function in the analysis. The user data analysis unit 112B determines that the value of the element data is away (deviate) from the target value (i) in a case in which the slope of the linear function obtained through the analysis is greater than the above-described threshold value 1 (increase) and a difference value at that point in time is greater than threshold value 4 (upper limit) or (ii) in a case in which the slope of the linear function is smaller than the above-described threshold value 2 (decrease) and the difference value at that point in time is smaller than threshold value 3 (lower limit). The user data analysis unit 112B determines that the value of the element data is close to (approaches) the target value (iii) in a case in which the slope of the linear function obtained through the analysis is greater than threshold value 1 (increase) and a difference value at that point in time is smaller than threshold value 3 (lower limit) or (iv) in a case in which the slope of the linear function is smaller than threshold value 2 (decrease) and the difference value at that point in time is greater than threshold value 4 (upper limit). The user data analysis unit 112B determines that the value of the element data is steady (unchanged) with respect to the target value (v) in a case in which the slope of the linear function is equal to or greater than threshold value 2 and smaller than or equal to threshold value 1.

The user data analysis unit 112B generates the element data change information indicating whether the value of the element data reaches the target value, the value of the element data deviates from the target value, the difference from the target value is unchanged, or the value of the element data approaches the target value, and outputs the generated component data change information to the keyword determination unit 113B.

In this embodiment, two types of keyword data 1 and keyword data 2 are stored in the data storage unit 118 in advance. Keyword data 1 and keyword data 2 are used in a case in which the value of component data does not reach the target value and a case in which the value of component data reaches the target value, respectively. Both of keyword data 1 and keyword data 2 include the element data, the keyword, and the category, which are associated with each other. Further, the keyword includes the base keyword and the additional keyword, which are associated with each other. The additional keyword is determined for each change trend of the element data value with respect to the target value. In keyword data 1, the trend-specific additional keyword is determined for each of the deviation, unchanged, and approach, whereas in keyword data 2, the trend-specific additional keyword is determined for each of the deviation and unchanged. An example of the keyword data will be described below.

The element data change information from the user data analysis unit 112B is input to the keyword determination unit 113B. In a case in which the element data change information indicates that the value of the element data has reached the target value, the keyword determination unit 113B determines that the keyword data 1 is used. In a case in which the element data change information indicates that the value of the element data does not reach the target value, the keyword determination unit 113B determines that keyword data 2 is used.

The keyword determination unit 113B refers to the keyword data determined to be used (any one of keyword data 1 and 2) to determine the basic keyword corresponding to the element data, similar to the keyword determination units 113 and 113A. Further, the keyword determination unit 113B determines the category corresponding to the element data, and outputs search category information indicating the determined category to the information search unit 115A.

The keyword determination unit 113B refers to the keyword data to read additional keyword corresponding to the change trend with respect to the target value of the element data indicated by the element data change information input from the user data analysis unit 112B. The keyword determination unit 113B forms a keyword set that is a set of read additional keyword and the basic keyword associated with the additional keyword, and outputs search keyword information indicating the formed keyword set to the information search unit 115A.

(Example of Target Data)

Next, an example of the target data according to this embodiment will be described.

FIG. 18 is a diagram illustrating an example (target data Dt21) of the target data according to this embodiment.

The target data Dt21 includes a target value of sleep time (Sleep in minute), a target value of a body weight (Weight in kg), and a target value of a body fat percentage (fat in %). The target value is a value associated with the element data and the user. The user regarding the target data Dt21 is specified on the basis of the device identification information of the terminal device 21 that is a transmission source, or the above-described user registration data.

In a case in which there is no input of the target data from the user, the user data analysis unit 112B may determine the target value of another component data based on certain element data. For example, in a case in which input data indicating height and weight is input, the user data analysis unit 112B may calculate a BMI based on the height and the weight and determine the calculated BMI as a target value of the BMI. For example, when standard height and weight are given, the user data analysis unit 112B determines, as a target value of the BMI, threshold value 3 (lower limit) to be 18.5 and threshold value 4 (upper limit) to be 25.

Further, in the user data analysis unit 112B, body data such as a body weight or an abdominal circumference recommended by a public institution, a medical institution, or the like may be determined as a target value in advance. For example, in the user data analysis unit 112B, 85 cm or 90 cm is determined as threshold value 4 (upper limit) that is a target value of the abdominal circumference. The abdominal circumference equal to or greater than 85 cm or 90 cm is an abdominal circumference in which a man and a woman are recommended to be diagnosed in a medical institution in Japan. Therefore, the user data analysis unit 112B receives target data indicating the target value from a device of the institution over the network 41 each time the target value is changed. The user data analysis unit 112B may display information indicating that the target value is changed in a case in which the target value indicated by the received target data has been changed. Thus, even in a case in which the target value is changed over time, the user can recognize the change.

(Example of Keyword Data)

Next, an example of the keyword data according to this embodiment will be described.

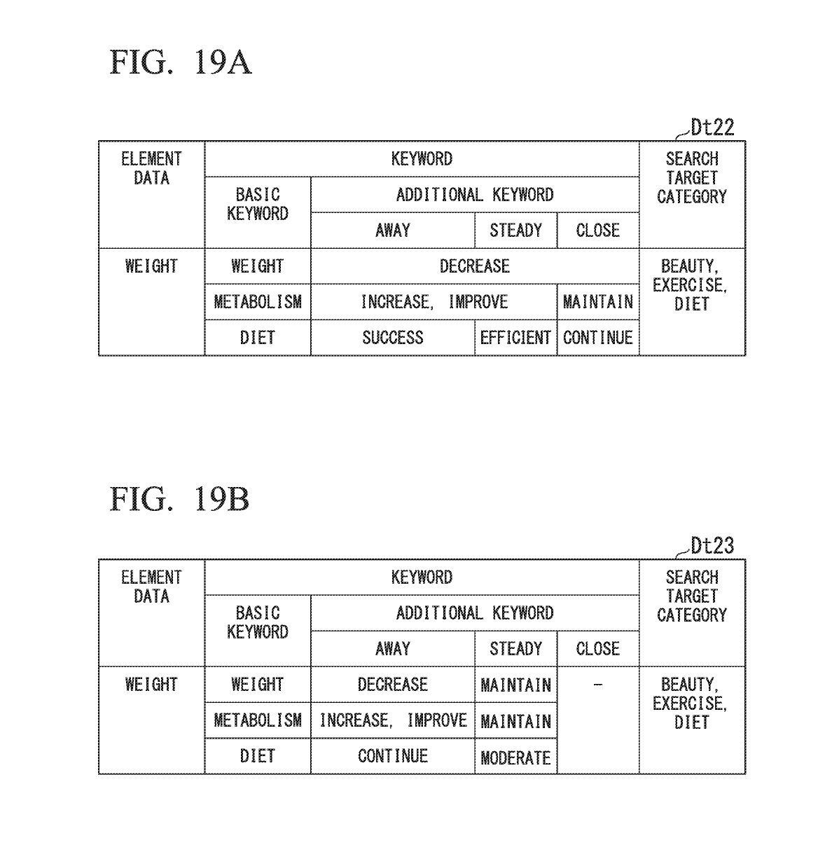

FIG. 19 is a diagram illustrating an example of the keyword data according to this embodiment.

FIGS. 19A and 19B illustrate keyword data Dt22 and Dt23, respectively. The keyword data Dt22 and Dt23 are keyword data 1 that is used in a case in which the value of the component data does not reach the target value, and keyword data 2 that is used when the value of the component data reaches the target value.

The element data included in each of the keyword data Dt22 and Dt23 is a body weight. The keyword data Dt22 and Dt23 include the basic keyword element data, the additional keyword, and the search target category, which are associated with each other. The keyword data Dt22 and Dt23 are the same in that basic keywords are “weight”, “metabolism”, and “diet”, and the search target category is “beauty”, “exercise”, and “diet”. However, in the keyword data Dt22, the trend-specific additional keywords are determined for “away” (deviation), “steady” (unchanged), and “close” (approach) as the change trends of the element data. On the other hand, in the keyword data Dt23, the trend-specific additional keywords are determined for “away” (deviation) and “steady” (unchanged) as the change trends of the element data, but is not determined for “close” (approach). This is because the element data does not further reach the target value in a case in which the element data has reached the target value.

The trend-specific additional keyword is associated with each basic keyword. For example, in the basic keyword “diet” in a second row of the keyword data Dt22 illustrated in FIG. 19A, “success”, “efficient”, and “continue” are associated with the change trends “away” (deviation), “steady” (unchanged), and “close” (approach) of the element data. Further, in the basic keyword “diet” in a second row of the keyword data Dt23 illustrated in FIG. 19B, “success” and “efficient” are associated with the change trends “continues” (deviation) and “moderate” (unchanged) of the element data.

Thus, as a set of basic keyword and trend-specific additional keyword, a phrase indicating a change trend of a thing indicated by a basic keyword according to the change trend with respect to the target value of the corresponding element data, and a phrase regarding maintenance and improvement of health according to the change trend of the element data may be used. Further, the set of basic keyword and trend-specific additional keyword may be a topic relating to a change trend with respect to the target value of the corresponding element data, the phrase regarding maintenance and improvement of health according to the change trend of the element data, or a phrase frequently included in the topic.

In the trend-specific additional keyword corresponding to the basic keyword “weight” in FIG. 19A, “decrease” is determined for any of the change trends “away” (deviation), “steady” (unchanged), and “close” (approach). This is because “decrease” is preferable even for any change trend in a case in which the element data “weight” does not reach the target value. Thus, according to the type of element data, a common trend-specific additional keyword may be used for any change trend in a case in which the element data does not reach the target value.

In other words, a phrase prompting the value of the element data as a trend-specific keyword to reach the target value is included in the keyword data Dt22 that is used in a case in which the value of the element data does not reach the target value. Thus, text information useful for the value of the element data to reach the target value is highly likely to be searched for, and irrelevant or less relevant information is less likely to be searched for. On the other hand, a phrase prompting the value of the element data as a trend-specific keyword to maintain the target value is included in the keyword data Dt23 that is used when the value of the element data reaches the target value. Thus, overview information useful for the value of the element data to maintain the target value is highly likely to be searched for, and irrelevant or less relevant information is less likely to be searched for.

Here, a process of the keyword determination unit 113B will be described in an example in which the element data information input from the user data analysis unit 112B indicates “weight”. Further, a case in which the element data change information that is input to the keyword determination unit 113B indicates that element data “weight” does not reach the target value and the change trend with respect to the target value of the “weight” indicates “approach” is assumed.

The keyword determination unit 113B determines that the keyword data Dt22 is used according to the fact that the element data “weight” does not reach the target value in the element data change information.

The keyword determination unit 113B refers to the keyword data Dt22 to read basic keywords “weight”, “metabolism” and “diet” corresponding to the element data information “weight”.

The keyword determination unit 113B reads each of the basic keywords “weight”, “metabolism”, and “diet” and the trend-specific additional keywords “decrease”, “maintain” and “continue” corresponding to the change trend “close” (approach) indicated by the element data change information.

The keyword determination unit 113B forms keyword sets “weight/reduce”, “metabolism/maintain”, and “diet/continue” that are sets of each of the read basic keywords “weight”, “metabolism”, and “diet” and each of the trend-specific additional keywords “decrease”, “maintain” and “continue” corresponding thereto. The keyword determination unit 113B outputs search keyword information indicating each of the formed keyword sets to the information search unit 115A.

Further, the keyword determination unit 113B refers to the keyword data Dt22 to read category information “beauty”, “exercise”, and “diet” corresponding to the element data information “weight”, and outputs the read category information “beauty”, “exercise”, and “diet” as search category information to the information search unit 115A.

The information search unit 115A generates search request information including the basic keyword “weight” and the trend-specific additional keyword “decrease” that form each keyword set “weight-decrease” indicated by the search keyword information (AND search) for each keyword set (OR search) and instructing to search for text information of the corner belonging to at least one of the categories “beauty”, “exercise”, and “diet” indicated by the search category information (OR search). Therefore, a sum set between keyword sets of text information of the corner including the basic keyword and the corresponding trend-specific additional keyword that form each keyword set is searched for. The sum set that has been searched for is limited by the category indicated by the search category information.

(Data Processing)

Next, an example of data processing according to this embodiment will be described.

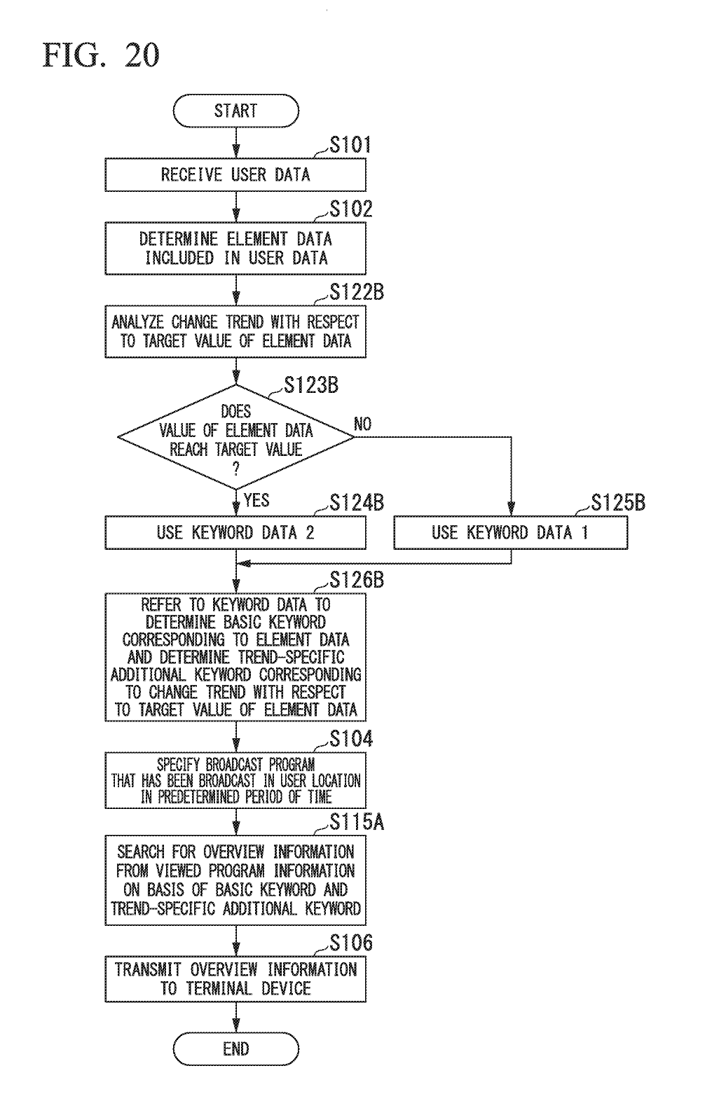

FIG. 20 is a flowchart illustrating an example of data processing according to this embodiment.

The data processing according to this embodiment includes steps S101, S102, S122B to S126B, S104, S115A, and S106.

In this embodiment, the user data analysis unit 112B executes the process of step S102, and the information search unit 115A executes the process of step S106.

In the data process illustrated in FIG. 20, the process proceeds to step S122B after the process of steps S101 and S102 ends.

(Step S122B) The user data analysis unit 112B analyzes a change trend with respect to a target value over time of the value of the element data included in the received user data, and determines whether the value of the element data reaches the target value, a difference from the target value is unchanged, or reaches the target value. The process then proceeds to step S123B.

(Step S123B) The user data analysis unit 112B determines whether the value of the element data reaches the target value. In a case in which it is determined that the value of the element data reaches the target value (YES in step S123B), the process proceeds to step S124B. In a case in which it is determined that the value of the element data does not reach the target value (NO in step S123B), the process proceeds to step S125B.

(Step S124B) The keyword determination unit 113B determines that keyword data 2 among keyword data 1 and keyword data 2 stored in the data storage unit 118 in advance is used. The process then proceeds to step S126B.

(Step S125B) The keyword determination unit 113B determines that keyword data 1 among keyword data 1 and keyword data 2 stored in the data storage unit 118 in advance is used. The process then proceeds to step S126B.

(Step S126B) The keyword determination unit 113B refers to the keyword data determined to be used, to determine a basic keyword corresponding to the element data. Further, the keyword determination unit 113B determines a trend-specific additional keyword corresponding to a change trend with respect to the target value of the element data. The keyword determination unit 113B forms a keyword set of basic keyword and additional keyword that have been determined. Further, the keyword determination unit 113B determines a category corresponding to the element data. The process then proceeds to step S104. After the process of steps S104, S115A, and S106 ends, the process illustrated in FIG. 20 ends.

As described above, in the data processing device 11B according to this embodiment, the state information acquisition unit (for example, user data analysis unit 112B) acquires the target value of the state information. Further, the data processing device 11B includes the state analysis unit (for example, user data analysis unit 112B) that analyzes a relationship between the state information and the target value of the state information acquired by the state information acquisition unit. Further, in the data processing device 11B, the keyword data stored in the keyword storage unit (for example, data storage unit 118) includes the second keyword corresponding to the relationship between the state information and the target value of the state information. Further, in the data processing device 11B, the keyword determination unit (for example, keyword determination unit 113B) further selects the second keyword corresponding to the relationship analyzed by the state analysis unit.

With this configuration, the text information including a phrase regarding the state of the body indicated by the state information received from the terminal device 21 and a phrase regarding a relationship between the state and the target value thereof as keywords is searched for. As the text information that has been searched for, text information suitable for the relationship between the state of the body of the user and the target value thereof is acquired, and text information having low relevance to the relationship with the target value of the state of the body of the user is excluded. Therefore, it is possible to improve a possibility of information on desired health according to the relationship between the state of the body and the target value thereof being obtained. For example, the user can more easily obtain information useful for approach to the target value.

(Modification Example)

The embodiments have been described above, but specific configurations are not limited to those described above, and various design modifications can be made without departing from the gist of the present invention.

In the above embodiment, the case in which the data processing devices 11, 11A, and 11B transmit the text information regarding the program broadcast on the previous day to the terminal device 21 at a predetermined time (for example, 10:00) mainly at one-day intervals has been described by way of example, but the present invention is not limited thereto. The data processing devices 11, 11A, and 11B may transmit the text information two times or more a day. The data processing devices 11, 11A, and 11B, for example, may transmit text information regarding a program broadcast from 17:00 of a previous day to 9:00 on the day at 10:00 every day, and transmit text information regarding a program broadcast from 9:00 to 16:00 of the day at 17:00 every day.

When the user data analysis units 112, 112A, and 112B receive the request signal from the terminal device 21, the user data analysis units 112, 112A, and 112B may read the user data and the user registration data that have been newly stored, from the user database server 12, and execute the above-described data processing (FIGS. 10, 16, and 20). The terminal device 21 receives text information from the data processing devices 11, 11A, and 11B (on-demand transmission) each time the terminal device 21 transmits the request signal in response to the operation of the user.

Further, the user database server 12 may transmit the newly received user data and user data to the data processing devices 11, 11A, and 11B each time the user database server 12 receives the user data from the terminal device 21. In this case, the data processing devices 11, 11A, and 11B may execute the above-described data processing (FIGS. 10, 16, and 20) for the user data and the user data received from the user database server 12. The terminal device 21 receives the text information from the data processing devices 11, 11A, and 11B (sequential transmission) each time the terminal device 21 transmits the user data.

The terminal device 21 may include a measurement unit that measures an index indicating the state of the body of the user described above. Accordingly, the terminal device 21 and the measurement device 22 are configured integrally as one terminal device. Further, the terminal device 21, the measurement device 22, and the reception device 23 may be configured integrally as one terminal device.

The case in which the terminal device 21 includes the display unit 214 has been described by way of example in the above description, but the present invention is not limited thereto. The display unit 214 may be an entity separate from the terminal device 21. Further, the terminal device 21 may include an alarm unit (for example, a speaker) in place of the display unit 214 or together with the display unit 214. In this case, the information output unit 217 performs a speech synthesis process on the text information to generate an audio signal, and outputs the generated audio signal to the alarm unit.

In a case in which the measurement device 22 is configured as an entity separate from the terminal device 21, the measurement device 22 may directly transmit the measurement data generated by the own device to the user database server 12 without passing through the terminal device 21. In this case, the terminal device 21 can omit the reception of the measurement data from the measurement device 22 and the transmission of the measurement data to the user database server 12.

The reception device 23 may receive the program data over the network 41. Further, in a case in which the terminal device 21 is a television reception device or in a case in which the terminal device 21 has a function of receiving and displaying the program data of the television broadcasting, the terminal device 21 may generate the viewing data, similar to the reception device 23, and transmit the generated viewing data to the viewing data database server 14.

The case in which the user database server 12 that stores the user data and the user registration data, the program information database server 13 that stores the program information, and the viewing data database server 14 that stores the viewing data are independent server devices has been described by way of example in the above-described embodiment, but the present invention is not limited thereto. Two or all of the user database server 12, the program information database server 13, and the viewing data database server 14 may be configured as an integrated device. Further, the data storage unit 118 of the data processing device 11, 11A, or 11B may store any one, any combination, or all of the user data and the user registration data, the program information, and the viewing data. Accordingly, the server device according to the data stored in the data storage unit 118 is integrated with the data processing devices 11, 11A, and 11B. For example, in a case in which the viewing data is stored in the data storage unit 118, the viewing data database server 14 according to the viewing data is integrated with the data processing device 11, 11A, or 11B, and the viewing data database server 14 as a separate server device may be omitted.

The information search unit 115 or 115A of the data processing device 11, 11A, or 11B may perform fuzzy search on the basis of the keyword determined in the keyword determination unit 113, 113A or 113B. The fuzzy search includes, for example, synonym search and associative word search. In a case in which synonym search or associative word search is performed, synonym data or associative word data is stored in the data storage unit 118 in advance. The synonyms data is data in which other semantically similar words (synonyms) are associated with each word. The associative word data is data in which other terms (associative words) indicating a thing or a concept associated from each word is associated with each word. Therefore, the keyword determination unit 113, 113A, or 113B refers to the synonyms data or the associative word data to specify the synonym or the associative word corresponding to the keyword determined in the above-described embodiment and includes the specified synonym or the associative word in the search keyword. The keyword determination unit 113A or 113B performs specifying of the synonym or the associative word on each of the basic keyword and the trend-specific additional keyword.

Further, prohibited phrase data indicating prohibited phrase may be stored in the data storage unit 118 of the data processing device 11, 11A, or 11B in advance. In this case, the keyword determination unit 113, 113A, or 113B refers to the prohibition phrase data to specify the prohibited phrase included in the keyword determined in the above-described embodiment, and excludes the specified prohibited phrase from the search keyword.

The information search unit 115A of the data processing device 11A or 11B may instruct the program information database server 13 to perform a proximity operation in search for the text information using each keyword set. For example, the information search unit 115A may impose a restriction that an interval between the basic keywords and the trend-specific additional keyword is within a predetermined interval threshold value (for example, five words or one statement). Thus, since the basic keywords and the trend-specific additional keyword approach each other, text information including a statement having a semantic relationship is searched for between the basic keywords and the trend-specific additional keyword. Therefore, it is possible to reduce or exclude text information including a statement having no semantic relationship between the basic keywords and the trend-specific additional keyword.

Further, the information search unit 115A of the data processing device 11, 11A, or 11B may impose a restriction that an order of the basic keywords and the trend-specific additional keyword is a predetermined order (for example, the basic keyword is earlier than the trend-specific additional keyword) to search for the text information using each keyword set. Accordingly, text information including the basic keywords and the trend-specific additional keyword in which a predetermined meaning or causal relationship is caused due to use in a predetermined order is searched for. Therefore, even when a predetermined basic keyword and trend-specific additional keyword are included, it is possible to reduce or exclude the text information having no a predetermined meaning or causal relationship.

The information search unit 115 or 115A of the data processing device 11, 11A, or 11B may instruct the program information database server 13 to search for the text information without imposing a restriction according to the broadcast region. Thus, narrowing of the information based on the region information is omitted. In this case, the program specifying unit 114 may be omitted.

The information search unit 115 or 115A of the data processing device 11, 11A, or 11B may adjust the presence or absence of a restriction of the search range, and the search range so that the number of cases of text information that have been searched for is within a predetermined range (for example, 20 cases or less). Restriction of the search range includes execution of the proximity operation, restriction using an order of the basic keyword and the trend-specific additional keyword, and restriction of a broadcasting region. The adjustment of the search range includes presence or absence of execution of the above-described fuzzy search, adjustment of the range of the broadcasting time that is a search target, and adjustment of the category of the broadcast program. The information search unit 115 or 115A transmits the text information of which the number of cases has been adjusted to the terminal device 21.

In the text information, an amount per corner may exceed a predetermined amount of information (for example, full-width 200 characters). In this case, the information search unit 115 or 115A of the data processing device 11, 11A, or 11B may perform a compression processing (summary) on the text information. Further, in a case in which a plurality of statements in which a phrase or a structure of a portion is the same are included in the received text information, the information search unit 115 or 115A may perform a combination process on the plurality of statements.

The program data may be stored in association with the program information in the program information database server 13. The program data is data of a broadcast video or audio. In this case, the information search unit 115 or 115A of the data processing devices 11, 11A, or 11B may instruct the program information database server 13 to search for the program data of the corner corresponding to the text information. The information search unit 115 or 115A may receive the program data corresponding to the text information from the program information database server 13, and transmit the received program data to the terminal device 21. Thus, the user of the terminal device 21 can view the corner corresponding to the acquired text information.

Although the case in which content is the broadcast program divided in units of corners has been described by way of example in the above-described embodiment, the present invention is not limited thereto. The content may be content divided by a predetermined period of time. Further, the content may be content that includes a video, an audio, text, and the like that can be transmitted from a device connected to the network 41.

A combination or all of the embodiments and the modification examples described above may be arbitrarily combined and implemented. For example, in the data processing device 11B according to the second embodiment, the user data analysis unit 112B may analyzes the change trend for the element data of which the target value is not determined, similar to the user data analysis unit 112A. The keyword determination unit 113B may specify the base keyword and the trend-specific additional keyword on the basis of the change trend analyzed by referring to the keyword data Dt11, similar to the keyword determination unit 113A.

A portion or all of the above-described data processing device 11, 11A, or 11B, such as the user data analysis unit 112, 112A, or 112B, the keyword determination unit 113, 113A, or 113B, the program specifying unit 114, the information search unit 115 or 115A, and a portion of the terminal device 21 such as the user data processing unit 216 and the information output unit 217 may be realized by a computer. In this case, the device may be realized by recording a program for realizing a control function thereof on a computer-readable recording medium, loading the program recorded on the recording medium into a computer system, and executing the program. Here, the “computer system” is a computer system embedded in the data processing device 11, 11A, and 11B, and the terminal device 21, which includes an OS or hardware such as a peripheral device. Further, the “computer-readable recording medium” refers to a portable medium such as a flexible disk, a magneto-optical disc, a ROM, or a CD-ROM, or a storage apparatus such as a hard disk embedded in the computer system. Further, the “computer-readable recording medium” may also include a recording medium that dynamically holds a program for a short period of time, such as a communication line when the program is transmitted over a network such as the Internet or a communication line such as a telephone line or a recording medium that holds a program for a certain period of time, such as a volatile memory inside a computer system including a server and a client in such a case. Further, the program may be a program for realizing some of the above-described functions or may be a program capable of realizing the above-described functions in combination with a program previously stored in the computer system.

Further, in this embodiment, a portion or all of the data processing device 11, 11A or 11B, and the terminal device 21 in the above-described embodiment may be realized as an integrated circuit such as a large scale integration (LSI). Each functional block of the data processing device 11, 11A, or 11B, and the terminal device 21 may be individually configured as a processor, or a portion or all thereof may be integrated and configured as a processor. Further, an integrated circuit scheme is not limited to the LSI and the block may be realized as a dedicated circuit or a general-purpose processor. Further, in a case in which an integrated circuit technology with which the LSI is replaced appears with the advance of a semiconductor technology, an integrated circuit according to the technology may be used.

### INDUSTRIAL APPLICABILITY

The present invention the present invention can be applied to a data processing device, a data processing system, a terminal device, a data processing method, and a data processing program, and particularly, to the field of health care.

### REFERENCE SIGNS LIST

1, 1A, 1B: data processing system

11, 11A, 11B: data processing device

111: communication unit

112, 112A, 112B: user data analysis unit

113, 113A, 113B: keyword determination unit

114: program specifying unit

115, 115A: information search unit

118: data storage unit

12: user database server

13: program information database server

14: viewing data database server

21: terminal device

211: user data input unit

212: measurement data input unit

213: operation data input unit

214: display unit

215: communication unit

216: user data processing unit

217: information output unit

218: data storage unit

22: measurement device

23: reception device

41: network

1. A data processing device, comprising:
a state information acquisition unit that acquires state information indicating a state of a body;
an information search unit that searches for information on broadcast content corresponding to the state information acquired by the state information acquisition unit from a content information storage unit that stores the information on the broadcast content; and
an output unit that outputs information on the broadcast content searched for by the information search unit.

2. The data processing device according to claim 1, further comprising:
a keyword storage unit that stores keyword data including a keyword regarding state information indicating the state of the body; and
a keyword determination unit that determines a keyword corresponding to the state information acquired by the state information acquisition unit from the keyword data stored in the keyword storage unit,
wherein the information search unit searches for information on the broadcast content corresponding to the state information from the content information storage unit on the basis of the keyword determined by the keyword determination unit.

3. The data processing device according to claim 1,
wherein the information on the broadcast content is information on the broadcast program.

4. The data processing device according to claim 3,
wherein the information output by the output unit is a portion of the information on the broadcast program.

5. The data processing device according to claim 1,
wherein the state information acquisition unit acquires first state information indicating a state of a first body, and second state information indicating a state of a second body, and
the information search unit searches for information on the broadcast content corresponding to the first state information and information on the broadcast content corresponding to the second state information.

6. The data processing device according to claim 1,
wherein the state information acquisition unit acquires a target value of the state information from the terminal device,
the data processing device further comprises a state analysis unit that analyzes a relationship between the state information and the target value of the state information acquired by the state information acquisition unit, and
the information search unit searches for information on the broadcast content corresponding to the relationship analyzed by the state analysis unit.

7. A data processing system comprising:
a terminal device that inputs state information indicating a state of a body; and
a data processing device,
wherein the data processing device further includes:
a state information acquisition unit that acquires the state information transmitted by the terminal device;
an information search unit that searches for information on broadcast content corresponding to the state information acquired by the state information acquisition unit from a content information storage unit that stores the information on the broadcast content; and
an output unit that outputs information on the broadcast content searched for by the information search unit.

8. A terminal device, comprising:
a state information input unit that inputs state information indicating a state of a body;
a communication unit that transmits the state information and receives information on broadcast content corresponding to the state information; and
an information output unit that outputs information on the broadcast content received by the communication unit.

9. The terminal device according to claim 8, further comprising:
a state information measurement unit that measures at least a portion of the state information.

10. A data processing method in a data processing device, comprising:
a state information acquisition step of acquiring state information indicating a state of a body;
an information search step of searching for information on broadcast content corresponding to the state information acquired by the state information acquisition unit from a content information storage unit that stores the information on the broadcast content; and
an output step of outputting information on the broadcast content searched for in the information search step.

11. A data processing program for causing a computer of a terminal device to execute:
a state information input procedure of inputting state information indicating a state of a body;
a communication procedure of transmitting the state information input in the state information input procedure and receiving information on broadcast content corresponding to the state information; and
an information output procedure of outputting information on the broadcast content received in the communication procedure.

12. The data processing device according to claim 2,
wherein the information on the broadcast content is information on the broadcast program.

13. The data processing device according to claim 5,
wherein the state information acquisition unit acquires a target value of the state information from the terminal device,
the data processing device further comprises a state analysis unit that analyzes a relationship between the state information and the target value of the state information acquired by the state information acquisition unit, and
the information search unit searches for information on the broadcast content corresponding to the relationship analyzed by the state analysis unit.
